(19) United States
(12) Patent Application Publication
(10) Pub. No.: US 2009/0181037 A1
(43) Pub. Date: Jul. 16, 2009

(54) Semi-Synthetic GLP-1 Peptide-FC Fusion Constructs, Methods and Uses

(76) Inventor: George Heavner, Radnor, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/263,738

(22) Filed: Nov. 3, 2008

Related U.S. Application Data
(60) Provisional application No. 60/984,862, filed on Nov. 2, 2007.

(51) Int. Cl. A61K 39/395; C07K 16/00; A61P 3/00; A61P 3/10

(52) U.S. Cl. 424/178.1; 530/391.1

(57) ABSTRACT

The invention relates to semi-synthetic biologic molecules which are conjugates of GLP-1 peptides and human multimeric proteins or protein fragments, such as an antibody Fc joined by a non-peptidyl bond. The constructs demonstrate biological activity and are useful making therapeutic compositions and therapeutic formulations for use in treating diseases characterized by lack of glycemic control.

### CROSS REFERENCE TO RELATED APPLICATIONS

This application claims priority to U.S. Provisional Application No. 60/984,862, filed 2 Nov. 2007, the entire contents of which is incorporated herein by reference.

### BACKGROUND OF THE INVENTION

1. Field of the Invention

The invention relates to compositions comprising a bioactive peptide chemically linked to immunoglobulin Fc proteins. More specifically, the invention relates to specific insulinotropic compositions comprising constructs of GLP-1 peptide and modified peptide analogs chemically linked to an antibody Fc.

2. Description of the Related Art

Recombinant proteins are an emerging class of therapeutic agents. Such recombinant therapeutics have engendered advances in protein formulation and chemical modification. Such modifications can potentially enhance the therapeutic utility of therapeutic proteins, such as by increasing half lives (e.g., by blocking their exposure to proteolytic enzymes), enhancing biological activity, or reducing unwanted side effects. One such modification is the use of immunoglobulin fragments fused to receptor proteins, such as enteracept. Therapeutic proteins have also been constructed using the Fc domain to attempt to provide a longer half-life or to incorporate functions such as Fc receptor binding, protein A binding, and complement fixation.

Diabetes is a growing epidemic that is estimated to affect over 300 million people by the year 2025 pending an effective pharmaceutical cure. Type 2 diabetes accounts for 90-95% of all cases. Complications resulting from sustained elevated plasma glucose levels include cardiovascular disease, nephropathy, neuropathy, and retinopathy. In addition, the β-cells of the pancreas die and therefore cease to secrete insulin during the later stages of type 2 diabetes. Current treatments for diabetes are associated with a variety of deleterious side effects including hypoglycemia and weight gain. In addition, current treatments for type 2 diabetes do not cure the disease but simply prolong the time until patients require insulin therapy.

Glucagon like peptide-1 (GLP-1) is a 37-amino acid peptide secreted from the L-cells of the intestine following an oral glucose challenge. A subsequent endogenous cleavage between the 6th and 7th position produces the biologically active GLP-1 (7-37) peptide. The GLP-1 (7-37) peptide sequence can be divided into 2 structural domains. The amino terminal domain of the peptide is involved in signaling while the remainder of the peptide appears to bind to the extracellular loops of the GLP-1 receptor in a helical conformation. In response to glucose, the active GLP-1 binds to the GLP-1 receptor on the pancreas and causes an increase in insulin secretion (insulinotropic action). In addition, it has been shown that GLP-1 reduces gastric emptying which decreases the bolus of glucose that is released into the circulation and may reduce food intake. These actions in combination lower blood glucose levels. GLP-1 has also been shown to inhibit apoptosis and increase proliferation of the β-cells in the pancreas. Thus, GLP-1 is an attractive therapeutic to lower blood glucose and preserve the β-cells of the pancreas of diabetic patients. In addition, GLP-1 activity is controlled by blood glucose levels. When blood glucose levels drop to a certain threshold level, GLP-1 is not active. Therefore, there is no risk of hypoglycemia associated with treatment involving GLP-1.

The viability of GLP-1 therapy has been demonstrated in the clinic. A six-week GLP-1 infusion lowered fasting and 8-hour mean plasma glucose levels effectively in type 2 diabetic patients. GLP-1 therapy also resulted in an improvement in β-cell function. Exenatide is a GLP-1 analogue currently in clinical trials. Exenatide was first identified in the saliva of the gila monster lizard, and is 53% identical to GLP-1. Exenatide can bind the GLP-1 receptor and initiate the signal transduction cascade responsible for the numerous activities that have been attributed to GLP-1 (7-37). To date, it has been shown to reduce HbA1c levels and serum fructosamine levels in patients with type 2 diabetes. In addition, it delayed gastric emptying and inhibited food intake in healthy volunteers.

However, GLP-1 is rapidly inactivated in vivo by the protease dipeptidyl-peptidase IV (DPP-IV). Therefore, the usefulness of therapy involving GLP-1 peptides has been limited by their fast clearance and short half-lives. For example, GLP-1 (7-37) has a serum half-life of only 3 to 5 minutes. GLP-1 (7-36) amide has a time action of about 50 minutes when administered subcutaneously. Even analogs and derivatives that are resistant to endogenous protease cleavage, do not have half-lives long enough to avoid repeated administrations over a 24 hour period. For example, exenatide is resistant to DPP-IV, yet it still requires twice daily preprandial dosing because of the short half-life and significant variability in in vivo pharmacokinetics. NN2211, another compound currently in clinical trials, is a lipidated GLP-1 analogue. It is expected to be dosed once daily.

Fast clearance of a therapeutic agent is inconvenient in cases where it is desired to maintain a high blood level of the agent over a prolonged period of time since repeated administrations will then be necessary. Furthermore, a long-acting compound is particularly important for diabetic patients whose past treatment regimen has involved taking only oral medication. These patients often have an extremely difficult time transitioning to a regimen that involves multiple injections of medication. A GLP-1 therapy that has an increased half-life would have a significant advantage over other GLP-1 peptides and compounds in development.

A large number of GLP-1 analogs with substitutions, deletions and modifications at various positions have been disclosed. See for example Buckley, D. I., Habener, J. F., Mallory, J. B. and Mojsov, S. GLP-1 analogs useful for diabetes treatment WO 91/11457. In addition, a number of hybrid peptides have been prepared. Hybrid peptides containing segments of GLP-1, glucagon and exendin-4, a high affinity GLP-1 receptor agonist isolated from the salivary gland of the Gila monster (Heloderma suspectum) with a 53% homology to GLP-1, have been prepared for multiple receptor binding. The addition of a glucagon sequence at the N-terminus blocks DPP-IV cleavage. C-terminal PEGylation through an introduced cysteine did not result in loss of activity.

A version of GLP-1 with an improved half-life was prepared by linking a maleimide group through a triethyleneglycol to the epsilon amino group of a lysine added to the C-terminus. In vivo this associates with albumin and forms a covalent bond with albumin cysteine.

With the intention of prolonging half life, fusion proteins have been prepared recombinantly comprising GLP-1 or analogs where the C-terminal carboxylic group of the peptide is fused to the N-terminal amino acid of an IgG4 through a Gly-Ser-rich linker. Both albumin and Fc fusion constructs have been disclosed with GLP-1 and analogs.

Therefore, there is a need in the art for compositions with GLP-1 activity for treatment of T2D which have prolonged serum half lives. Several approaches to creating such a molecule based on the natural GLP-1 peptide sequence including protease resistant analogs and derivatives or conjugates incorporating a lipid, a hydrophilic polymer such as PEG, or, as discussed, a immuoglobulin fusion construct.

The approach of fusing a GLP-1 peptide or analog to an immunoglobulin Fc is thus one way of prolonging half-life of the peptide. However, because such fusion proteins are expressed recombinantly, they are limited to containing the 20 natural mammalian amino acids. Although there are techniques whereby unnatural amino acids can be incorporated into proteins by manipulation of the genetic code, these methods are restricted to the use of N^{α}-L-amino acids. There are numerous analogs of biologically active peptides that contain D-amino acids, N^{β}-amino acids or higher homologues, non-amino acid moieties and cyclic peptides using non-cysteine side chains. In addition, some biologically active peptides require a free carboxylic acid group for activity. None of these structures can be incorporated into fusion proteins using produced by recombinant techniques.

### SUMMARY OF THE INVENTION

The present invention relates to GLP-1 peptide semi-synthetic immunoglobulin Fc fusion proteins that can incorporate all of the above-described variants of GLP-1 analog peptides not accessible via recombinant techniques. Not only is biological activity retained for active GLP-1 peptides, but the unique presentation of two peptides on the immunoglobulin Fc scaffold can show biological activity that is not predicted or explained by the current understanding of structure-activity relationships of GLP-1.

Accordingly, in one aspect the invention relates to a bioactive conjugate for medical use comprising: a GLP-1 peptide or analog thereof conjugated by a non-peptidyl linkage to an antibody Fc fragment. The bioactive therapeutic is conjugated to the antibody Fc fragment directly or indirectly through a covalent bond to an oxidized amino acid moiety of the antibody Fc wherein the reactive carbonyl is an aldehyde or a ketone. In another aspect of the bioactive conjugate, the covalent bond linking the GLP-1 peptide directly or indirectly to the Fc is formed by reaction of a nucleophilic group selected from the group consisting of a primary amine, hydrazine, acyl hydrazide, carbazide, semicarbazide and thiocarbazide with the reactive carbonyl-containing moiety of the Fc.

The invention further relates to compositions of the general formula

B-(L)ₙ-(F) (I)

where B represents an at least one bioactive GLP-1 peptide, variant or derivative, F represents an antibody Fc comprising the structure (X)ₘ-(D)ₚ-CH2-CH3 where X represents any naturally occurring amino acid which may be incorporated and produced by standard molecular biological engineering techniques, where m is an integer from 0-20, D is a multimerizing or dimerizing domain such as at least a portion of an immunoglobulin hinge region, p is an integer from 0 to 1 and CH2 represents at least a portion of an immunoglobulin CH2 constant region which is joined to at least a portion of an immunoglobulin CH3 constant region. L represents a linker comprising a polymeric structure which is substantially nonimmunogenic and provides a flexible linkage between the bioactive moiety and F, allowing the construct to have alternative orientations, where n can be the integers 0 or 1. Where n is 0, the linkage between B and F is a non-peptidyl covalent bond. When n is 1, the linkage between L and F is a non-peptidyl bond. In one embodiment, L is comprised of poly(alkylene oxide) residues such as polyethylene glycol. The resulting construct can, optionally, be further linked to the same or other polypeptides, polymers, labels, radioisotopes, or actives by association or covalent linkage, such as, but not limited to, a Cys-Cys disulfide bond.

In particular embodiments, the invention includes conjugates of formula I comprising compounds of the formulae:

B—F (II) and multimers thereof where the C-terminus of B is attached to the N-terminus of F or where the N-terminus of B is attached to the N-terminus of F and F lacks the dimerizing domain;
B-L-F (III) and multimers thereof wherein F is an Fc domain lacking the dimerizing domain and is attached by the N-terminus to L, and L is further attached at an alternated site to the C-terminus of B; or wherein F is a polypeptide as described capable of forming an Fc domain and is attached by the N-terminus to L, and L is further attached at an alternated site to the N-terminus of B; and
B¹—F—B² (IV) where B¹ and B² are the same or different GLP-1 peptides or are conjugated to F via alternative sites on the same GLP-1 and where F has the dimerizing domain; and
B¹-L¹-F-L²-B² (V) where B¹ and B² are the same or different GLP-1 peptides or are conjugated to L¹ and L², respectively, via alternative sites on B1 and B2 and where F has the dimerizing domain. In each case, either the link between B and F or the link between L and F is a non-peptidyl bond.

Compositions in which a GLP-1 conjugate is multimerized with an immunoglobulin heavy chain which is not conjugated to a GLP-1 are also encompassed by the compositions. Such compositions, such as a monovalent composition, may be the result of association of the conjugates of formulas I-V with a free heavy chain covalently or non-covalently or by monovalent conjugation of already associated heavy chains as in a pre-formed Fc region.

In one embodiment, the present invention relates to a method for chemically modifying a GLP-1 peptide to increase the serum half-life thereof, in a site-specific manner without reducing the biological activity of the molecule. The method of the invention comprises the steps of forming a reactive carbonyl at the N-terminus of antibody Fc and conjugating the GLP-1 peptide thereto through a non-peptidyl bond. In one embodiment, the method involves conjugating the GLP-1 peptide to a polymer, such as a PEG polymer, having a first and second site for conjugation where the GLP-1 peptide is bound to a first site, and binding the second conjugation site of the PEG having a nucleophilic functional group with an aldehyde present on an antibody Fc at the N-terminus, the aldehyde generated by oxidative cleavage of an N-terminal serine or threonine residue as expressed by recombinant protein technology.

### BRIEF DESCRIPTION OF THE DRAWINGS

FIG. 1 contains graphs showing the activity of bioactive GLP-1 peptide 1 compared to wild-type GLP-1 peptide in a bioactivity assay wherein GLP-1 Receptor binding stimulated cAMP is measured in INS-1E cells contacted with the test article.

FIG. 2 is a graph showing the relative cAMP stimulatory activity of Peptide 3 as compared to wild-type GLP-1 peptide in the cAMP assay as in FIG. 2.

FIG. 3 is a graph showing the activity of several versions of GLP-1 peptide-Fc conjugate as measured by a cAMP assay.

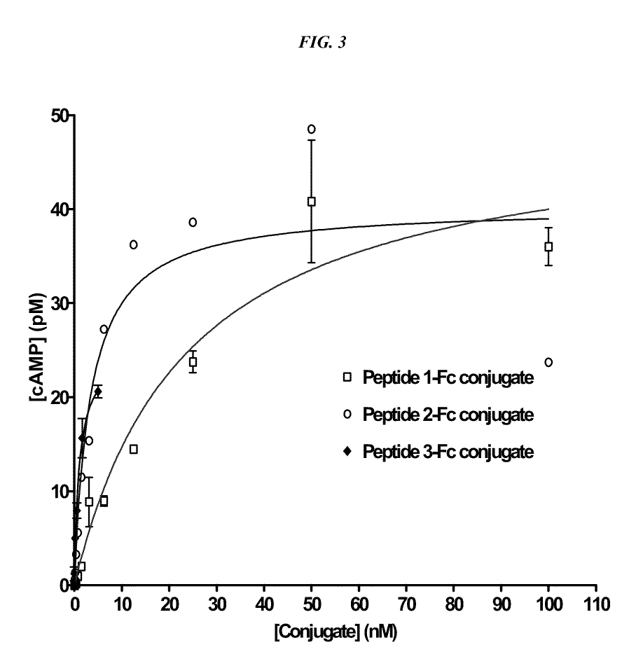

### DETAILED DESCRIPTION OF THE INVENTION

### Abbreviations

FMOC: 9-fluorenylmethoxycarbonyl; Boc: t-butoxycarbonyl; GLP-1 glucagons-like peptide-1;

### DEFINITIONS

The term “conjugate” is intended to refer to the entity formed as a result of covalent attachment of a molecule, such as a biologically active peptide, to a antibody Fc by a hydrazone or semicarbazone linkage formed by reaction with an reactive carbonyl on the Fc which may be via the incorporation of a synthetic polymer molecule, such as poly(ethylene glycol), therebetween and when present the polymer is attached to the Fc by a hydrazone or semicarbazone linkage formed by reaction with an reactive carbonyl on the Fc.

The terms “Fc,” “Fc-containing protein” or “Fc-containing molecule” as used herein refer to a monomeric, dimeric or heterodimeric protein having at least an immunoglobulin CH2 and CH3 domain. The CH2 and CH3 domains can form at least a part of the dimeric region of the protein molecule (e.g., antibody) when functionally linked to a dimerizing or multimerizing domain such as the antibody hinge domain. The Fc portion of the antibody molecule (fragment crystallizable, or fragment complement binding) denotes one of the well characterized fragments produced by digestion of an antibody with various peptidases, in this case pepsin. While various antibody fragments are defined in terms of the digestion of an intact antibody, one of skill will appreciate that such Fc fragments may be synthesized de novo either chemically or by utilizing recombinant DNA methodology, peptide display, or the like. The CH2- and CH3 domains are preferably derived from human germline sequences such as those disclosed in WO2005005604.

As used herein, the terms “peptide and “protein” are used interchangeably to refer to a polymer of amino acid residues linked together by peptide bonds. The term is meant to include proteins, polypeptides, and peptides of any size, structure, or function. Typically, however, a peptide will be at least six amino acids long and a protein will be at least 50 amino acids long. A peptide typically has a molecular weight up to about 10,000 Da, while peptides having a molecular weight above that are commonly referred to as proteins. Modifications of the peptide side chains may be present, along with glycosylations, hydroxylations, and the like. Additionally, other non-peptidic molecules, including lipids and small drug molecules, may be attached to the polypeptide. A protein may be naturally occurring, recombinant, or synthetic, or any combination of these. A peptide may also be a fragment of a naturally occurring protein or peptide. A protein may be a single molecule or may be a multi-molecular complex. The term protein may also apply to amino acid polymers in which one or more amino acid residues is an artificial chemical analogue of a corresponding naturally occurring amino acid. An amino acid polymer in which one or more amino acid residues is an “unnatural” amino acid, not corresponding to any naturally occurring amino acid, is also encompassed by the use of the terms “peptide and “protein” herein.

By “increase in serum half-life” or “increased t_{1/2}” is meant the positive change in circulating half-life of a modified biologically active molecule relative to its non-modified form. Serum half-life is measured by taking blood samples at various time points after administration of the biologically active molecule, and determining the concentration of that molecule in each sample. Measuring the change in serum concentration with time allows calculation of the serum half-life. By comparing the serum half-life of a modified molecule, e.g. conjugated molecule, with an unmodified molecule, the relative increase in serum half-life or t_{1/2} may be determined. The increase is desirably at least about two-fold, but a smaller increase may be useful.

The term “fusion protein” refers to a protein composed of two or more polypeptides that, although typically unjoined in their native state, are joined by their respective amino and carboxyl termini through a peptide linkage to form a single continuous polypeptide. It is understood that the two or more polypeptide components can either be directly joined or indirectly joined through a sequence of one or more amino acids which acts as a spacer and may provide flexibility.

The terms “functional group”, “active moiety”, “reactive site”, “chemically reactive group” and “chemically reactive moiety” are used in the art and herein to refer to distinct, definable portions or units of a molecule. The terms are somewhat synonymous in the chemical arts and are used herein to indicate the portions of molecules that perform some function or activity and are reactive with other molecules. The term “active,” when used in conjunction with functional groups, is intended to include those functional groups that react readily with electrophilic or nucleophilic groups on other molecules, in contrast to those groups that require strong catalysts or highly impractical reaction conditions in order to react (i.e., “non-reactive” or “inert” groups). For example, as would be understood in the art, the term “active ester” would include those esters that react readily with nucleophilic groups such as amines. Exemplary active esters include N-hydroxysuccinimidyl esters or 1-benzotriazolyl esters. Typically, an active ester will react with an amine in aqueous medium in a matter of minutes, whereas certain esters, such as methyl or ethyl esters, require a strong catalyst in order to react with a nucleophilic group. As used herein, the term “functional group” includes protected functional groups.

The term “protected functional group” or “protecting group” or “protective group” refers to the presence of a moiety (i.e., the protecting group) that prevents or blocks reaction of a particular chemically reactive functional group in a molecule under certain reaction conditions. The protecting group will vary depending upon the type of chemically reactive group being protected as well as the reaction conditions to be employed and the presence of additional reactive or protecting groups in the molecule, if any. Protecting groups known in the art can be found in Greene, T. W., et al., Protective Groups In Organic Synthesis, 3rd ed., John Wiley & Sons, New York, N.Y. (1999).

The term “linkage” or “linker” (L) is used herein to refer to an atom or a collection of atoms used to link, preferably by one or more covalent bonds, interconnecting moieties such as two polymer segments or a terminus of a polymer and a reactive functional group present on a bioactive agent, such as a polypeptide.

By “residue” is meant the portion of a molecule remaining after reaction with one or more molecules. For example, an amino acid residue in a polypeptide chain is the portion of an amino acid remaining after forming peptide linkages with adjacent amino acid residues. The glyoxylyl functional group is the residue formed by periodiate treatment of an N-terminal serine or threonine on a polypeptide.

As used herein, a “GLP-1 peptide,” or “GLP-1 peptide, variant, or derivative” can be at least one GLP-1 peptide, GLP-1 fragment, GLP-1 homolog, GLP-1 analog, or GLP-1 derivative. A GLP-1 peptide has from about twenty-five to about forty-five naturally occurring or non-naturally occurring amino acids that have sufficient homology to native GLP-1 (7-37) such that they exhibit insulinotropic activity by binding to the GLP-1 receptor on β-cells in the pancreas. GLP-1 (7-37) has the amino acid sequence HAEGTFTSDVSSYLEGQAAKEFIAWLVKGRG (SEQ ID NO: 1).

A GLP-1 fragment is a polypeptide obtained after truncation of one or more amino acids from the N-terminus and/or C-terminus of GLP-1 (7-37) or an analog or derivative thereof. A GLP-1 homolog is a peptide in which one or more amino acids have been added to the N-terminus and/or C-terminus of GLP-1 (7-37), or fragments or analogs thereof. A GLP-1 analog is a peptide in which one or more amino acids of GLP-1 (7-37) have been modified and/or substituted. A GLP-1 analog has sufficient homology to GLP-1 (7-37) or a fragment of GLP-1 (7-37) such that the analog has insulinotropic activity. A GLP-1 derivative is defined as a molecule having the amino acid sequence of a GLP-1 peptide, a GLP-1 homolog or a GLP-1 analog, but additionally having chemical modification of one or more of its amino acid side groups, α-carbon atoms, terminal amino group, or terminal carboxylic acid group.

### GLP-1 Peptides

Numerous active GLP-1 fragments, analogs and derivatives are known in the art and any of these analogs and derivatives can also be part of the GLP-1 mimetibody of the present invention. Some GLP-1 analogs and GLP-1 fragments known in the art are disclosed in U.S. Pat. Nos. 5,118,666, 5,977,071, and 5,545,618, and Adelhorst, et al., J. Biol. Chem. 269:6275 (1994). Examples include, but not limited to, GLP-1 (7-34), GLP-1 (7-35), GLP-1 (7-36), Gln9-GLP-1 (7-37), D-Gln9-GLP-1 (7-37), Thr16-Lys18-GLP-1 (7-37), and Lys18-GLP-1 (7-37). Any GLP-1 compound can be part of the heterologous fusion proteins of the present invention as long as the GLP-1 compound itself is able to bind and induce signaling through the GLP-1 receptor. GLP-1 receptor binding and signal transduction can be assessed using in vitro assays such as those described in EP 619,322 and U.S. Pat. No. 5,120,712, respectively.

Numerous active GLP-1 fragments, analogs and derivatives are known in the art and any of these analogs and derivatives can also be part of the heterologous fusion proteins of the present invention. Some examples of novel GLP-1 analogs as well as GLP-1 analogs and derivatives known in the art are provided herein.

To prevent degradation and inactivation by DPP-IV, numerous analogs of GLP-1 have been prepared including N^{α}-methyl-His¹, α-methyl-His¹, desamino-His¹ and imidazole-lactic-acid-GLP-1 were prepared (Sarrauste de Menthière, et al. 2004 Eur. J. Med. Chem. 39: 473-480). All of these except α-methyl His¹-GLP-1 were stable in the presence of DPP IV in vitro. They all showed receptor affinity and in vitro biological activity comparable to native GLP-1 in RINm5F cells. Only desamino-His¹-GLP-1 showed a 15-fold loss of receptor affinity compared to native GLP-1. All analogues stimulated intracellular cAMP production in RINm5F cells in concentrations comparable to GLP-1.

Another approach to preventing degradation by DPP-IV is to modify or replace selected amino acid residues in the natural GLP-1 sequence. In another example, the N-terminal amino group may be acylated, e.g. acetylated, to prevent recognition and cleavage by DPP-IV.

Non-limiting examples of suitable GLP-1 peptides, variants and derivatives for this invention appear as SEQ ID NO: 2: His-Xaa2-Xaa3-Gly-Xaa5-Xaa6-Xaa7-Xaa8-Xaa9-Xaa10-Xaa11-Xaa12-Xaa13-Xaa14-Xaa15-Xaa16-Xaa17-Xaa18-Xaa19-Xaa20-Xaa21-Phe-Xaa23-Xaa24-Xaa25-Xaa26-Xaa27-Xaa28-Xaa29-Xaa30-Xaa31, wherein: Xaa2 is Ala, Gly, Ser, Thr, Leu, Ile, Val, Glu, Asp, or Lys; Xaa3 is Glu, Asp, or Lys; Xaa5 is Thr, Ala, Gly, Ser, Leu, Ile, Val, Arg, His, Glu, Asp, or Lys; Xaa6 is Phe, His, Trp, or Tyr; Xaa7 is Thr or Asn; Xaa8 is Ser, Ala, Gly, Thr, Leu, Ile, Val, Glu, Asp, or Lys; Xaa9 is Asp or Glu; Xaa10 is Val, Ala, Gly, Ser, Thr, Leu, Ile, Met, Tyr, Trp, His, Phe, Glu, Asp, or Lys; Xaa11 is Ser, Val, Ala, Gly, Thr, Leu, Ile, Glu, Asp, or Lys; Xaa12 is Ser, Val, Ala, Gly, Thr, Leu, Ile, Glu, Asp or Lys; Xaa13 is Tyr, Phe, Trp, Glu, Asp or Lys; Xaa14 is Leu, Ala, Met, Gly, Ser, Thr, Leu, Ile, Val, Glu, Asp or Lys; Xaa15 is Glu, Ala, Thr, Ser, Gly, Gln, Asp or Lys; Xaa16 is Gly, Ala, Ser, Thr, Leu, Ile, Val, Gln, Asn, Arg, Cys, Glu, Asp or Lys; Xaa17 is Gln, Asn, Arg, His, Glu, Asp or Lys; Xaa18 is Ala, Gly, Ser, Thr, Leu, Ile, Val, Arg, Glu, Asp or Lys; Xaa19 is Ala, Gly, Ser, Thr, Leu, Ile, Val, Met, Glu, Asp or Lys; Xaa20 is Lys, Arg, His, Gln, Trp, Tyr, Phe, Glu or Asp; Xaa21 is Glu, Leu, Ala, His, Phe, Tyr, Trp, Arg, Gln, Thr, Ser, Gly, Asp or Lys; Xaa23 is Ile, Ala, Val, Leu or Glu; Xaa24 is Ala, Gly, Ser, Thr, Leu, Ile, Val, His, Glu, Asp or Lys; Xaa25 is Trp, Phe, Tyr, Glu, Asp or Lys; Xaa26 is Leu, Gly, Ala, Ser, Thr, Ile, Val, Glu, Asp or Lys; Xaa27 is Val, Leu, Gly, Ala, Ser, Thr, Ile, Arg, Glu, Asp or Lys; Xaa28 is Lys, Asn, Arg, His, Glu or Asp; Xaa29 is Gly, Ala, Ser, Thr, Leu, Ile, Val, Arg, Trp, Tyr, Phe, Pro, His, Glu, Asp or Lys; Xaa30 is Arg, His, Thr, Ser, Trp, Tyr, Phe, Glu, Asp or Lys; and Xaa31 is Gly, Ala, Ser, Thr, Leu, Ile, Val, Arg, Trp, Tyr, Phe, His, Glu, Asp, Lys.

Another preferred group of GLP-1 peptides, variants or derivatives are exemplified in SEQ ID NO: 3: His-Xaa2-Xaa3-Gly-Thr-Xaa6-Xaa7-Xaa8-Xaa9-Xaa10-Ser-Xaa12-Tyr-Xaa14-Glu-Xaa16-Xaa17-Xaa18-Xaa19-Lys-Xaa21-Phe-Xaa23-Ala-Trp-Leu-Xaa27-Xaa28-Gly-Xaa30, wherein: Xaa2 is Ala, Gly, or Ser; Xaa3 is Glu or Asp; Xaa6 is Phe or Tyr; Xaa7 is Thr or Asn; Xaa8 is Ser, Thr or Ala; Xaa9 is Asp or Glu; Xaa10 is Val, Leu, Met or Ile; Xaa12 is Ser or Lys; Xaa14 is Leu, Ala or Met; Xaa16 is Gly, Ala, Glu or Asp; Xaa17 is Gln or Glu; Xaa18 is Ala or Lys; Xaa19 is Ala, Val, Ile, Leu or Met; Xaa21 is Glu or Leu; Xaa23 is Ile, Ala, Val, Leu or Glu; Xaa27 is Val or Lys; Xaa28 is Lys or Asn; and Xaa30 is Arg or Glu.

A group of exendin-4 peptides are given in SEQ ID NO:4 and having the formula His Xaa2 Xaa3 Gly Thr Phe Thr Xaa8 Asp Xaa10 Ser Lys Gln Xaa14 Glu Glu Glu Ala Val Arg Leu Xaa22 Xaa23 Glu Xaa25 Leu Lys Xaa28 Gly Gly Pro Ser Ser Gly Ala Pro Pro Pro-Z wherein Xaa2 is Ser, Gly or Thr, Xaa3 is Asp or Glu, Xaa8 is Ala, Ser, or Thr, Xaa10 is Leu, Ile, Val, pentylglycine or Met, Xaa14 is Ala, Leu, Ile, pentylglycine, Val, Xaa22 is Phe, Tyr or naphthylalanine, Xaa23 is Ile, Val, Leu, pentylglycine, tert-butylglycine or Met, Xaa25 is Ala, Trp, Phe, Tyr or naphthylalanine, Xaa28 is Ala or Asn, and Z is —OH or NH2 as disclosed in U.S. Pat. No. 7,223,725.

These peptides can be prepared by methods disclosed and/or known in the art. The Xaas in the sequence (and throughout this specification, unless specified otherwise in a particular instance) include specified amino acid residues, derivatives or modified amino acids thereof. Because the enzyme, dipeptidyl-peptidase IV (DPP-IV), may be responsible for the observed rapid in vivo inactivation of administered GLP-1, GLP-1 peptides, homologs, analogs and derivatives that are protected from the activity of DPP-IV are preferred.

The peptides may also comprise modified, non-naturally occurring and unusual amino acids substituted or added to their amino acid sequences. A list of exemplary modified, non-naturally occurring and unusual amino acids is provided below.

MODIFIED (UNUSUAL) AMINO ACID SYMBOL

| | |
|---|---|
| 2-Aminoadipic acid | Aad |
| 3-Aminoadipic acid | Baad |
| beta-Alanine, beta-Aminopropionic acid | bAla |
| 2-Aminobutyric acid | Abu |
| 4-Aminobutyric acid, piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | BAib |
| 2-Aminopimelic acid | Apm |
| 2,4-Diaminobutyric acid | Dbu |
| Desmosine | Des |
| 2,2′-Diaminopimelic acid | Dpm |
| 2,3-Diaminopropionic acid | Dpr |
| N-Ethylglycine | EtGly |
| N-Ethylasparagine | EtAsn |
| Hydroxylysine | Hyl |
| allo-Hydroxylysine | AHyl |
| 3-Hydroxyproline | 3Hyp |
| 4-Hydroxyproline | 4Hyp |
| Isodesmosine | Ide |
| allo-Isoleucine | AIle |
| N-Methylglycine, sarcosine | MeGly |
| N-Methylisoleucine | MeIle |
| 6-N-Methyllysine | MeLys |
| N-Methylvaline | MeVal |
| Norvaline | Nva |
| Norleucine | Nle |
| Ornithine | Orn |

Amino acids in a polypeptide that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as the ability to cause signal transduction when a cell bearing the cognate receptor is contacted with the polypeptide.

Such variants have an altered amino acid sequence and can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade that includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

It is generally preferred that the GLP-1 compound that is part of the fusion protein have no more than 6 amino acids that are different from the corresponding amino acid in GLP-1 (7-37), GLP-1 (7-36), or Exendin-4.

The bioactive peptides, linked to the alternate chains of the Fc, optionally with a linker moiety therebetween, may be the same or different. The bioactive peptides may be linked to intervening linker or to the Fc from any residue on the peptide so long as the final conjugate displays the desired bioactivity. Bioactivity may be measured by in vitro assays, for example binding activity, by in vivo activity such as in animal models of disease, or by the response of a subject following administration of the conjugate.

In a preferred embodiment, GLP-1 peptides can be synthesized that contain groups that can not be incorporated recombinantly. For example, activated peptide-linkers 1 and 2 below contain a D-amino acid in the second position of the GLP-1 peptide. This modification has been used to stabilize the peptide against the action of DPP-IV (dipeptidylpeptidase IV) that cleaves off an N-terminal peptide, inactivating GLP-1. All three peptides contain a short chain polyethyleneglycol group to act as a spacer to separate the peptide from the Fc.

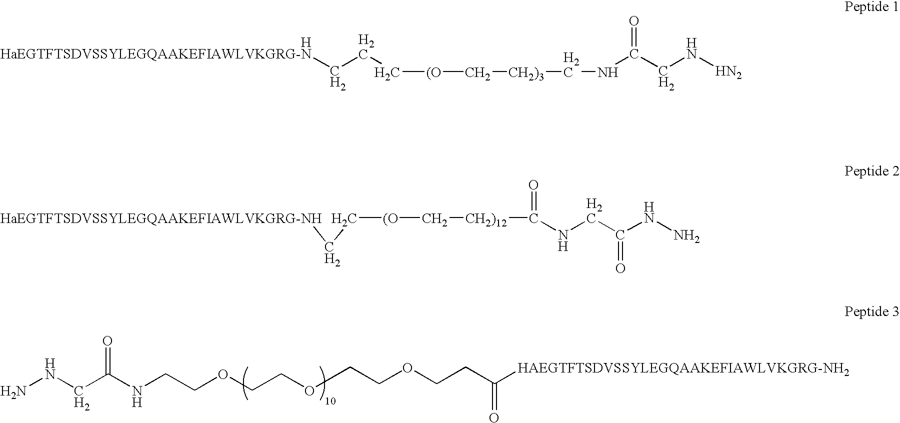

### Antibody Fc

In the present invention, the antibody Fc portion of the construct can be derived from an intact naturally occurring isolated antibody, an isolated monoclonal antibody, or be synthesized de novo recombinantly or chemically or by a combination of methods. An antibody Fc-region is conveniently prepared from an intact antibody, preferably a human antibody or antibody comprising human constant regions, by proteolytic cleavage of the heavy chain. Papain, a plant enzyme, which in the presence of a reducing agent such as cysteine, cleaves the human IgG1 molecule between the CH1 and CH2 domains of the heavy chain (between a His and a Thr) leaving threonine as the N-terminal residue. The histidine residue is the C-terminal position of abciximab when papain digestion is performed on the antibody known as c7E3 in the presence of cysteine. Prolonged treatment, or excessive amounts of papain, typically results in overdigestion of the Fc domain, although the Fab domains often remain resistant to degradation by papain. Thus, when recovery of the intact Fc-region is desired conditions must be carefully controlled. Fc region retain the ability to bind to protein A and can be purified using protein A affinity chromatography. A co-owned patent application (WO2007/024743), discloses that the Fc of glycosylated Abs are more resistant to papain digestion than the deglycosylated or aglycosylated or non-glycosylated Abs. Thus, if the Fc region is to be deglycosylated, that step should be performed following papain digestion.

In another embodiment, the Fc-may be synthesized by recombinant methods and be representative of any human class/subclass or of another species as desired. The sequences of human and animal immunoglobulin are accessible to the public in publications, e.g. Kabat, et al. Sequences of Proteins of Immunological Interest, U.S. Dept. Health (1983), or available online. Sequences derived from human germline information as well as known and useful therapeutic human antibody sequences and variations thereof have been summarized in WO2005005604 which is incorporated herein by reference. Particular reference is made to the sequences and variants of the human immunoglobulin class IgA₁, IgA₂, IgD, IgE, IgG₁, IgG2₁, IgG3₁, IgG24, and IgM heavy chains shown in sections of FIG. 32-40 and recited in SEQ ID NOS: 32-40 of WO2005005604 which provides a source of information for the synthesis of human antibody Fc regions.

The Fc-region or domain of an antibody imparts non-antigen binding functions to the antibody, termed “effector functions”, such as complement binding, antibody-dependent cell cytoxicity (ADCC), and other functions mediated through the binding of subregions of this dimeric structure with immune cell surface receptors, Fc-receptors. Certain natural and synthetic variants of the Fc-region polypeptides sequences with altered effector functions include the subclass variants; e.g. IgG₁, IgG2₁, IgG3₁, IgG24; and mutant polypeptides as described in e.g. U.S. Pat. Nos. 5,624,821, 6,528,624, 6737356, 7183387, and publication WO04099249A2.

While in large part, these functions contribute to the antibody neutralization capabilities by destroying target (antigen displaying) cells, these functions are also dependent on glycans attached to the Fc in the CH2 domain (see e.g. Jefferis & Lund. 2002. Immunology Letters. 82(1-2):57-65, 2002). Thus, when the Fc is stripped of glycans the killer functions are eliminated. Fc may are naturally produced when expressed recombinantly in a bacterial host cell or can be removed enzymatically using glycosidase enzymes when desired.

Where the Fc, comprising an Xₘ-D-CH2-CH3, is used, the natural amino acids of X provide structural flexibility by allowing the fusion protein to have alternative orientations and binding properties.

### Linkers

The linker is preferably made up of amino acids linked together by peptide bonds. Thus, in preferred embodiments, the linker is made up of from 1 to 20 amino acids linked by peptide bonds, wherein the amino acids are selected from the 20 naturally occurring amino acids. Some of these amino acids may be glycosylated, as is well understood by those in the art. In a more preferred embodiment, the 1 to 20 amino acids are selected from glycine, alanine, serine, proline, asparagine, glutamine, and lysine. Even more preferably, a linker is made up of a majority of amino acids that are sterically unhindered, such as glycine and alanine. Thus, preferred linkers are poly(Gly-Ser), polyglycines (particularly (Gly)₄, (Gly)₅), poly(Gly-Ala), and polyalanines. Other specific examples of linkers are: (Gly)₃Lys(Gly)₄ (SEQ ID NO: 5), (Gly)₃AsnGlySer(Gly)₂ (SEQ ID NO: 6), (Gly)₃Cys(Gly)₄ (SEQ ID NO: 7), and GlyProAsnGlyGly (SEQ ID NO: 8).

To explain the above nomenclature, for example, (Gly)₃Lys(Gly)₄ means Gly-Gly-Gly-Lys-Gly-Gly-Gly-Gly. Combinations of Gly and Ala are also preferred. The linkers shown here are exemplary; linkers within the scope of this invention may be much longer and may include other residues.

Non-peptide linkers are also possible. For example, alkyl linkers such as —NH—(CH₂)s-C(O)—, wherein s=2-20 could be used. These alkyl linkers may further be substituted by any non-sterically hindering group such as lower alkyl (e.g., C₁-C₆) lower acyl, halogen (e.g., Cl, Br), CN, NH2, phenyl, etc. An exemplary non-peptide linker is a PEG linker which has a molecular weight of 100 to 5000 kD, preferably 100 to 500 kD. The peptide linkers may be altered to form derivatives in the same manner as described above.

The linker is preferably a polymer. Suitable polymers include, for example, polyethylene glycol (PEG), polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and cellulose derivatives, including methylcellulose and carboxymethyl cellulose, starch and starch derivatives, polyalkylene glycol and derivatives thereof, copolymers of polyalkylene glycols and derivatives thereof, polyvinyl ethyl ethers, and α,β-Poly[(2-hydroxyethyl)-DL-aspartamide, and the like, or mixtures thereof. In one aspect of the invention the polymer is of defined chemical composition and molecular weight range. A preferred embodiment, the polymer is PEG.

The linkers may be comprised of one or more moieties selected from the group consisting of: a peptide of 1-20 amino acids, a polyethyleneglycol composed of 1-50 ethyleneoxy units and, optionally derivatized with terminal amino, hydroxyl, mercapto, and/or carboxy groups; polydispersed polyethyleneglycols of molecular weight 300-40,000 optionally derivatized with terminal amino, hydroxyl, mercapto, and/or carboxy groups; C₂₋₂₀ alkyl optionally derivatized with terminal hydroxyl, amino and/or carboxy groups; and substituted C₂₋₂₀ alkyl optionally derivatized with terminal hydroxyl, mercapto, amino and/or carboxy groups. The linking moiety comprising a suitable reactive nucleophile as described above may also be attached to the biologically generated carrier molecule, e.g. the antibody Fc, of the construct at the N-terminus prior to conjugation with the GLP-1 peptide.

The linker (L), when present in the conjugate of formula I, may include polymer chains or units that are biostable or biodegradable. For example, polymers with repeat linkages may have varying degrees of stability under physiological conditions depending on bond liability. Polymers with such bonds can be categorized by their relative rates of hydrolysis under physiological conditions based on known hydrolysis rates of low molecular weight analogs, e.g., from less stable to more stable polycarbonates (—O—C(O)—O—)> polyesters (—C(O)—O—)> polyurethanes (—NH—C(O)—O—)> polyorthoesters (—O—C((OR)(R′))—O—)> polyamides (—C(O)—NH—). Similarly, the linkage systems attaching a water-soluble polymer to a target molecule may be biostable or biodegradable, e.g., from less stable to more stable carbonate (—O—C(O)—O—)>ester (—C(O)—O—)>urethane (—NH—C(O)—O—)>orthoester (—O—C((OR)(R′))—O—)>amide (—C(O)—NH—). These bonds are provided by way of example, and are not intended to limit the types of bonds employable in the polymer chains or linkers of the soluble polymers of the invention.

Methods of Preparing the Conjugates

While the compositions of the invention may be prepared by any means known in the art or yet to be discovered, certain processes are provided herein which are particularly suited to the generation of the compositions.

### Preparing the Fc Protein and Preparation for Conjugation

The biologically generated multimerized carrier molecule, such as an antibody Fc comprising at least a part of the cysteine containing region known as the hinge is prepared from recombinantly expressed protein product which has been secreted in the multimerized (dimeric) form. Where mammalian host cells are used to express the protein, the endproduct may be glycosylated. Where prokaryotic host cells, e.g. E. coli, are used to express the protein, glycosylation normally does not occur.

If the protein contains an N- or O-linked glycosyl groups, the carbohydrate is susceptible to oxidation by oxidizing agents used to affect the formation of the N-terminal glyoxal group, producing additional reactive carbonyl species. Carbohydrates can be removed prior to chemical linking using PNGase followed by purification by hydrophobic interaction HPLC.

The N-terminal residue of the Fc-domain is prepared for conjugation reactions by creating a reactive (electrophilic) carbonyl in the residue prior to the first peptide bond of the N-terminus of at least one of the heavy chains of the Fc-domain. Reactive carbonyl configurations included as electrophilic moieties amenable to the methods of preparing the constructs of the present invention include aldeydes and ketones. Where a ketone is formed the terminal radical is selected from linear or branched lower alkyl composed of 1-6 carbons, substituted linear or branched lower alkyl composed of 1-6 carbons, aryl, or substituted aryl.

An antibody Fc may naturally comprise a serine or threonine or may be engineered by methods well know in the art, or chemically altered to display a serine or threonine at the N-terminus.

In one of the aspects of the method of the invention, the antibody Fc is a human IgG1-Fc having an N-terminal Thr which is the product of cleavage of any full-length human IgG1 antibody preparation with papain. The plant derived enzyme papain (E.C. 4.3.22.2) cleaves the heavy chain of the antibody above (N-terminal to) the hinge region which joins the two or more chains that form the Fc-region and between a His and Thr, thereby leaving the Thr as the N-terminal residue. In another aspect of the invention, the protease is selected from the group consisting of plasmin, pepsin, a matrix metalloproteinase including MMP-7, neutrophil elastase (HNE), stromelysin (MMP-3), macrophage elastase (MMP-12), trypsin, and chymotrypsin and other plant enzymes such as ficin (EC. 3.4.22.3) and bromolain (E.C. 3.4.4.24). Where other proteolytic enzymes are used, it is preferable to choose one that cleaves above the hinge domain, e.g. plasmin, HNE, or papain.

In one aspect, the GLP-1 peptide semi-synthetic immunoglobulin Fc fusion proteins may be prepared by the selective chemical oxidation with periodic acid of the N-terminal amino acid of an antibody Fc to produce an N-terminal glyoxylic acid (Scheme I). The glyoxylic acid does not react with amino groups under normal conditions. It does, however, react rapidly and selectively with hydrazines, carbhydrazones and oximes to generate a Schiffs base (i.e. —C═N—). This group is moderately stable to hydrolysis, giving release of the peptide, and can be made completely stable by gentle reduction with agents such as NaBH₃CN.

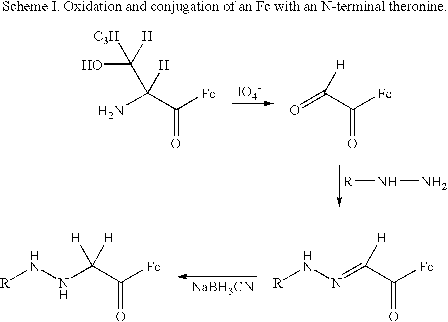

Thus, in the specific embodiment wherein the N-terminal residue of the biologically generated and mature polypeptide is a serine or threonine, a particularly useful method is the use of selective chemical oxidation with periodic acid to produce an N-terminal glyoxylic acid (Geoghegan and Stroh. 1992 Bioconjugate Chem 3: 138-146, and U.S. Pat. No. 5,362,852; Garnter, et al. 1996. Bioconjugate Chem. 7:38-44 and WO 98/05363 (Feb. 12, 1998). The 2-amino alcohol structure —CH(NH2)CH(OH)-exists in proteins and peptides in N-terminal Ser or Thr and in hydroxylysine. Its very rapid oxidation by periodate at pH 7 generates an aldehyde at the N-terminus. Therefore, the periodate method for preparation of a reactive carbonyl for site-directed attachment will be unique to the N-terminal position according to scheme II below.

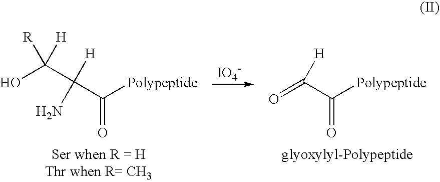

Only proteins containing an N-terminal threonine or serine can be oxidized with periodic acid to give N-terminal glycoxylic acid derivatives, however, synthesis of glyoxylyl peptides using an Fmoc-protected α,α′-diaminoacetic acid derivative (Fmoc-NH₂CHCO₂H) has been reported (Far and Melnyk. 2005. J Peptide Sci 11(7) 424-430). An N-terminal glycyl residue of a protein may be converted to an aldehyde by transamination, for example with glyoxylate under relatively mild conditions (Dixon, H. B. F. and Fields, R. 1979. Methods Enzymol. 25: 409-419). Another method of adding amino acid residues is by reverse proteolysis using proteolytic enzymes such as trypsin, carboxypeptidase Y as described by Rose (Rose, et al. 1983. Biochem J. 211:671-676).

The carbonyl group of an aldehyde or ketone may be reacted with amino groups under aqueous conditions in the presence of a reducing agent to give an amide. An aldehyde or ketone reacts rapidly and selectively under mild conditions with nucleophilic groups such as hydrazines, hydrazides and O-alkylhydroxylamines to generate a Schiff's base (i.e. —C═N—), azomethines, hydrazones and oximes, which can be made completely stable by reduction with agents such as NaBH₃CN.

Once the glyoxylyl-Fc, aldehyde-Fc, or keto-Fc is generated, it may be reacted with a nucleophilic moiety selected from the group consisting of an aminooxy, hydrazine, hydrazide or semicarbazide group on the other molecule to be conjugated (Fields and Dixon, (1968), Biochem. J. 108:883-887; Gaertner et al., (1992), Bioconjugate Chem. 3:262-268; Geoghegan and Stroh, (1992), Bioconjugate Chem. 3:138-146; Gaertner et al., (1994), J. Biol. Chem. 269:7224-7230). The term “hydrazine” includes hydrocarbyl derivatives of diazine (H₂NNH₂). When one or more substituents are acyl groups, the compound is a hydrazide. N-alkylidene derivatives are hydrazones having the structure R₂C═NNR₂. Hydrazones formally derive from aldehydes or ketones by replacing ═O by ═NNH2 (or substituted analogues). The reaction of the glyoxylyl group with a hydrazine will form a hydrazone.

Thus, in the present invention, a glyoxylyl-Fc (HCO—CO-Fc), a keto-Fc, or a simple aldehyde-Fc (HCO-Fc) may be reacted with an activated GLP-1 peptide having a hydrazine or hydrazide functionality to form a hydrazone at one or both of the N-termini of the Fc structure which may be further reduced to a hydrazine compound according to the following scheme (III) below:

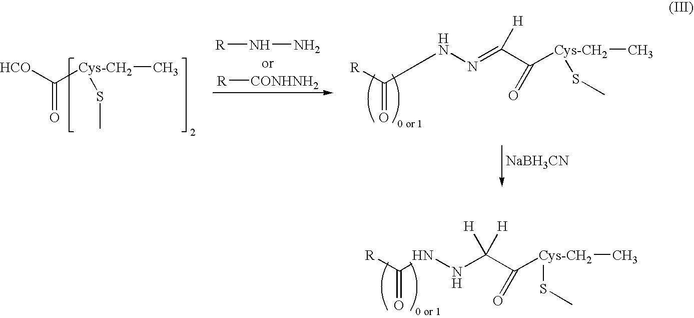

A second type of reaction with an aldehyde, including but not limited to glyoxylyl, or a ketone is known as the Wittig reaction. The Wittig reaction is a chemical reaction of an aldehyde or ketone with a triphenyl phosphonium ylide (often called a Wittig reagent) or with tri-n-butylphosphine to give an alkene and trialkylphosphine oxide (Wittig, G.; Schöllkopf, U. Ber. 1954, 87, 1318; Wittig, G.; Haag, W. Ber. 1955, 88, 1654).

It will be recognized that where the recombinant or natural isolated multimeric protein, such as an Fc, is used the conjugate will provide the option of a construct which is multivalent with respect to the bioactive peptide and, further, can be heteromeric with respect to the peptides attached to the multiple N-termini of the protein. In the example where an antibody Fc is used as the carrier molecule, the bioactive peptide may be attached to both of the N-terminal residues of the Fc-dimer. In another embodiment, only one bioactive peptide is attached to one of the N-termini of the antibody Fc. In yet another embodiment, two different bioactive peptides are attached to the N-termini of the antibody Fc creating a “bispecific” conjugate which may have “dual bioactivities”.

In the embodiments where the peptide is linked via the N-terminus, the peptide may be synthesized using standard solid phase chemistry. After assembly of the peptide on the resin and removal of the N-terminal protecting group, an appropriately protected bi-functional linker is coupled to the deprotected N-terminal amino group.

In another embodiment of an N-terminal linked peptide, the final coupling on the resin can incorporate a linker capable of reaction selectively with an aldehyde or ketone group, cleavage of the linker-peptide from the resin and removal of residual protecting groups will give the appropriate peptide composition for conjugation. Alternatively, after coupling with the peptide, the remaining functionality of the linker may be further derivatized to yield a functionality that will react with an aldehyde or ketone group. Similar strategies utilizing solution phase chemistry can be used to generate similar peptide compositions.

In embodiments where attachment of the peptide is desired via the C-terminus, the peptide may be synthesized by solid phase chemistry and cleaved from the resin by hydrazine or a hydrazine derivative followed by removal of protecting groups to yield the appropriately functionalized peptide. Where a linker is to be conjugated at the C-terminus, the linker may be directly coupled to the resin, followed by assembly of the peptide. The peptide-linker can be cleaved from the resin by hydrazine or a hydrazine derivative followed by removal of protecting groups to yield the appropriately functionalized peptide-linker. Alternatively the peptide may be prepared on a resin such as the Universal PEG NovaTag resin (Novabiochem). The Mmt group on the Universal PEG Novatag resin may then be removed to give a free amino group. This amino group may be derivatized to yield a functionality that will react with an aldehyde or ketone group. Alternatively, an appropriately protected bi-functional linker is coupled to the amino group. Similar strategies utilizing solution phase chemistry can be used to generate similar peptide compositions.

### Preparation of GLP-1 Peptides and Peptide Linker Constructs

The GLP-1 peptides of the compositions may be produced by either synthetic chemical processes or by recombinant methods or a combination of both methods. The GLP-1 peptides may be prepared as full-length polymers or be synthesized as non-full length fragments and joined. Chemical synthesis of peptides is routinely performed methods well known to those skilled in the art for either solid phase or solution phase peptide synthesis. For solid phase peptide synthesis, so called t-Boc (tert-Butyloxy carbonyl) and Fmoc (Fluorenyl-methoxy-carbonyl) chemistry, referring to the N-terminal protecting groups, on polyamide or polystyrene resin have become the conventional methods (Merrifield, R B. 1963 and Sheppard, R C. 1971, respectively). Unlike ribosomal protein synthesis, solid-phase peptide synthesis proceeds in a C-terminal to N-terminal fashion. The N-termini of amino acid monomers is protected by these two groups and added onto a deprotected amino acid chain. Deprotection requires strong acid such as trifluoroacetic acid for t-Boc and bases such as piperidine for Fmoc. Stepwise elongation, in which the amino acids are connected step-by-step in turn, is ideal for small peptides containing between 2 and 100 amino acid residues.

Non-naturally occurring residues are incorporated into a GLP-1 peptide or protein. Examples of non-ribosomally installed amino acids that may be used in accordance with a present invention and still form a peptide backbone include, but are not limited to: D-amino acids, β-amino acids, pseudo-glutamate, γ-aminobutyrate, ornithine, homocysteine, N-substituted amino acids (R. Simon et al., Proc. Natl. Acad. Sci. U.S.A. (1992) 89: 9367-71; WO 9119735 (Bartlett et al.), U.S. Pat. No. 5,646,285 (Baindur), α-aminomethyleneoxy acetic acids (an amino acid-Gly dipeptide isostere), and α-aminooxy acids and other amino acid derivatives having non-genetically non-encoded side chain function groups etc. Peptide analogs containing thioamide, vinylogous amide, hydrazino, methyleneoxy, thiomethylene, phosphonamides, oxyamide, hydroxyethylene, reduced amide and substituted reduced amide isosteres and β-sulfonamide(s) may be employed.

In another process, unnatural amino acids have been introduced into recombinantly produced proteins by a method of codon suppression. In one aspect, the use of codon suppression techniques could be adapted to introduce an aldehyde or ketone functional group or any other functional group in any suitable position within a polypeptide chain for conjugation (see e.g. Ambrx WO2006/132969).

Alternatively, recombinant expression methods are particularly useful. Recombinant protein expression using a host cell (a cell artificially engineered to comprise nucleic acids encoding the sequence of the peptide and which will transcribe and translate, and, optionally, secrete the peptide into the cell growth medium) is used routinely in the art. For recombinant production process, a nucleic acid coding for the amino acid sequence of the peptide would typically be synthesized by conventional methods and integrated into an expression vector. Such methods are particularly preferred for manufacture of the polypeptide compositions comprising the peptides fused to additional polypeptide sequences or other proteins or protein fragments or domains. The host cell can optionally be at least one selected from E. coli, COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof. Also provided is a method for producing at least one peptide, comprising translating the peptide encoding nucleic acid under conditions in vitro, in vivo or in situ, such that the peptide is expressed in detectable or recoverable amounts. The techniques well known in the art, see, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989).

### Preparation of the Conjugates

Once the Fc polypeptide comprising an N-terminal aldehyde or ketone has been isolated, the GLP-1 molecule comprising a suitable nucleophilic group is reacted with the Fc-region under aqueous conditions and the conjugate isolated. Thus, the GLP-1 molecule may comprise the linker L as defined herein. In the alternative, the moiety to serve as the linker L in Formula I, may comprise a nucleophile capable of reacting with an aldehyde or ketone under aqueous conditions and be conjugated to the Fc-region, which conjugate is subsequently joined to the GLP-1 molecule by any suitable manner known in the art.

In one embodiment of the method of the invention, the general process for conjugation of proteins or peptides as anticipated by this invention includes the following steps: (1) incorporation of a first reactive group on the GLP-1 moiety, (2) reaction of the GLP-1 moiety with a polymer that forms a covalent linkage with the first reactive component on the GLP-1 moiety, (3) reacting the polymer with the oxidized antibody Fc to form a covalently linked GLP-1-Fc conjugate, and (4) purification of the GLP-1-Fc conjugate. Where the incorporation of the first reactive group on the on the GLP-1 moiety in the case of a GLP-1 peptide may be a reactive group present within a residue of the peptide, e.g. the free carboxyl group, the free alpha-amino group of the N-terminus, or a reactive sidegroup of a residue within the peptide chain.

The attachment of a linker to the previously synthesized full-length GLP-1 peptides of the present invention can proceed by any method taught in the art. The activation of the GLP-1 peptide for conjugation to the Fc-domain can be conveniently accomplished during the solid phase synthetic process using tri-Boc-hydrazinoacetic acid at the final residue prior to cleavage from the resin to give the hydrazine derivatized peptide or, in the alternative, the GLP-1 peptide may be conjugated to a linker as described above, which linker will comprise a functional group capable of reaction with the reactive carbonyl of the Fc.

Several methods for site-directed or selective attachment of PEG have been described. For example, WO 99/45026 suggests chemical modification of a N-terminal serine residue to form an aldehyde functionality suitable for reaction with a polymer terminated with a hydrazide or semicarbazide functionality. U.S. Pat. Nos. 5,824,784 and 5,985,265 suggest reacting a polymer bearing a carbonyl group with the amino terminus of a protein under reducing alkylation conditions and at a pH that promotes selective attack at the N-terminus. WO 99/03887 and U.S. Pat. Nos. 5,206,344 and 5,766,897 relate to the site-directed PEGylation of cysteine residues that have been engineered into the amino acid sequence of proteins (cysteine-added variants). These methods offer advantages over non-specific attachment as the domains and structure of the GLP-1 peptide may be preserved by strategically linking the polymer a residue distal from the functional portion of the peptide. Alternatively, a reactive group may be introduced on the peptide, preferably at a site which preserves the biological activity of the peptide, following preparation of the peptide by either recombinant or chemical synthetic processes.

A “reactive group” or “functional group” is a an arrangement of atoms that can, under appropriate conditions, react with a second chemical functional group to cause a covalent bond between the species displaying the first functional group and that displaying the second functional group. For example, activating groups reactive with an amine (—NR₂) include electrophilic groups such as tosylate, mesylate, halo(chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)).

The chemically reactive functional group is selected from the group consisting of a primary or secondary amine, hydroxyl, thiol, carboxyl, aldehyde, and a ketone.

### Methods of Testing the Compositions

The conjugates of the invention retain or display the desired bioactivity which can be tested, assayed or measured by any in vitro, in vivo or by any surrogate marker or response appropriate and as will be known to those skilled in the art. For example, a typical desired bioactivity is protein-protein interaction, e.g. ligand binding or target binding, which is easily measured by solid phase capture assays such as ELISA. In other cases, the bioactivity will be measured as the result of contact of the conjugate with the target protein or cell, such as by measuring receptor activation which may be quantitatied by the effect that it has on intracellular processes such as Ca2+ release or intracellular cAMP concentration. In yet a more complex method of testing the conjugate, the downstream effect on a cell, organ, or animal may be observed.

In the case of semi-synthetic GLP-1 analog conjugates of the present invention, all effects related to natural or “wild-type” GLP-1 can be used to ascertain the bioacitivity of the conjugates. Such measurements include GLP-1 receptor binding, GLP-1 receptor activation as indicated by intracellular cAMP, measure of the amount or change in insulin secretion by, e.g. isolated pancreatic islets or whole pancreas or as measured in the serum of an animal after administration of the conjugate. The GLP-1 receptor is a G protein-coupled receptor (GPCR) that shares sequence identity with other “Family B” receptors such as those for secretin, glucagon, and vasoactive intestinal peptide. Another method for monitoring GLP-1 bioactivity is a pancreatic duct cell differentiation assay as taught in (Liu et al. 2004. Cell Biol. Internat. 28:69-73).

### Pharmaceutical Preparations and Articles of Manufacture

In another aspect, the invention relates to semi-synthetic GLP-1 Fc fusion constructs, as described herein, which are modified by the covalent attachment of an organic moiety. Such modification can produce a GLP-1 protein with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified mimetibodies and ligand-binding fragments of the invention can comprise one or more organic moieties that are covalently bonded, directly or indirectly, to the GLP-1 mimetibody or specified portion or variant. Each organic moiety that is bonded to a GLP-1 mimetibody or ligand-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, a GLP-1 mimetibody modified by the covalent attachment of polylysine is encompassed by the invention. Hydrophilic polymers suitable for modifying mimetibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the GLP-1 mimetibody of the invention has a molecular weight of about 800 to about 150,000 Daltons as a separate molecular entity. For example, PEG₂₅₀₀, PEG₅₀₀₀, PEG₇₅₀₀, PEG₉₀₀₀, PEG₁₀₀₀₀, PEG₁₂₅₀₀, PEG₁₅₀₀₀, and PEG_{20,000}, wherein the subscript is the average molecular weight of the polymer in Daltons, can be used.

The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying mimetibodies of the invention can be saturated or can contain one or more units of unsaturation. Fatty acids that are suitable for modifying mimetibodies of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include monoesters of dicarboxylic acids that comprise a linear or branched lower alkyl group. The lower alkyl group can comprise from one to about twelve, preferably one to about six, carbon atoms.

GLP-1 protein compositions of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990. Pharmaceutically acceptable carriers can be routinely selected that are suitable for the mode of administration, solubility and/or stability of the GLP-1 mimetibody composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/GLP-1 mimetibody or specified portion or variant components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

GLP-1 mimetibody compositions can also include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts such as citrate.

Additionally, the GLP-1 mimetibody or specified portion or variant compositions of the invention can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the GLP-1 mimetibody compositions according to the invention are known in the art, e.g., as listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), the disclosures of which are entirely incorporated herein by reference. Preferred carrier or excipient materials are carbohydrates (e.g., saccharides and alditols) and buffers (e.g., citrate) or polymeric agents.

As noted above, the invention provides for stable formulations, which can preferably include a suitable buffer with saline or a chosen salt, as well as optional preserved solutions and formulations containing a preservative as well as multi-use preserved formulations suitable for pharmaceutical or veterinary use, comprising at least one GLP-1 mimetibody or specified portion or variant in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

Optionally, one or more pharmaceutically-acceptable anti microbial agents may be added. Meta-cresol and phenol are preferred pharmaceutically-acceptable microbial agents. One or more pharmaceutically-acceptable salts may be added to adjust the ionic strength or tonicity. One or more excipients may be added to further adjust the isotonicity of the formulation. Glycerin is an example of an isotonicity-adjusting excipient. Pharmaceutically acceptable means suitable for administration to a human or other animal and thus, does not contain toxic elements or undesirable contaminants and does not interfere with the activity of the active compounds therein.

A pharmaceutically-acceptable salt form of the GLP-1 fusion proteins of the present invention may be used in the present invention. Acids commonly employed to form acid addition salts are inorganic acids such as hydrochloric acid, hydrobromic acid, hydriodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Preferred acid addition salts are those formed with mineral acids such as hydrochloric acid and hydrobromic acid. Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

Pharmaceutical Uses and Methods of Administration

Administration may be via any route known to be effective by the physician of ordinary skill. Peripheral, parenteral is one such method. Parenteral administration is commonly understood in the medical literature as the injection of a dosage form into the body by a sterile syringe or some other mechanical device such as an infusion pump. Peripheral parenteral routes can include intravenous, intramuscular, subcutaneous, and intraperitoneal routes of administration.

The heterologous fusion proteins of the present invention may also be amenable to administration by oral, rectal, nasal, or lower respiratory routes, which are non-parenteral routes. Of these non-parenteral routes, the lower respiratory route and the oral route are preferred.

The fusion proteins of the present invention can be used to treat a wide variety of diseases and conditions. The fusion proteins of the present invention primarily exert their biological effects by acting at a receptor referred to as the “GLP-1 receptor.” Subjects with diseases and/or conditions that respond favorably to GLP-1 receptor stimulation or to the administration of GLP-1 compounds can therefore be treated with the GLP-1 fusion proteins of the present invention. These subjects are said to “be in need of treatment with GLP-1 compounds” or “in need of GLP-1 receptor stimulation”. Included are subjects with non-insulin dependent diabetes, insulin dependent diabetes, stroke (see WO 00/16797), myocardial infarction (see WO 98/08531), obesity (see WO 98/19698), catabolic changes after surgery (see U.S. Pat. No. 6,006,753), functional dyspepsia and irritable bowel syndrome (see WO 99/64060). Also included are subjects requiring prophylactic treatment with a GLP-1 compound, e.g., subjects at risk for developing non-insulin dependent diabetes (see WO 00/07617). Subjects with impaired glucose tolerance or impaired fasting glucose, subjects whose body weight is about 25% above normal body weight for the subject's height and body build, subjects with a partial pancreatectomy, subjects having one or more parents with non-insulin dependent diabetes, subjects who have had gestational diabetes and subjects who have had acute or chronic pancreatitis are at risk for developing non-insulin dependent diabetes.

An “effective amount” of a GLP-1 compound is the quantity which results in a desired therapeutic and/or prophylactic effect without causing unacceptable side effects when administered to a subject in need of GLP-1 receptor stimulation. A “desired therapeutic effect” includes one or more of the following: 1) an amelioration of the symptom(s) associated with the disease or condition; 2) a delay in the onset of symptoms associated with the disease or condition; 3) increased longevity compared with the absence of the treatment; and 4) greater quality of life compared with the absence of the treatment. For example, an “effective amount” of a GLP-1 compound for the treatment of diabetes is the quantity that would result in greater control of blood glucose concentration than in the absence of treatment, thereby resulting in a delay in the onset of diabetic complications such as retinopathy, neuropathy or kidney disease. An “effective amount” of a GLP-1 compound for the prevention of diabetes is the quantity that would delay, compared with the absence of treatment, the onset of elevated blood glucose levels that require treatment with anti-hypoglycemic drugs such as sulfonyl ureas, thiazolidinediones, insulin and/or bisguanidines.

The dose of fusion protein effective to normalize a patient's blood glucose will depend on a number of factors, among which are included, without limitation, the subject's sex, weight and age, the severity of inability to regulate blood glucose, the route of administration and bioavailability, the pharmacokinetic profile of the fusion protein, the potency, and the formulation.

While having described the invention in general terms, the embodiments of the invention will be further disclosed in the following examples.

### Example 1

### Preparation of Activated GLP-1 Peptides

### Example 1A

### (His-[D-Ala]-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-(N-3-aminopropyl)-PEG3—NH—CO—CH₂—NH—NH₂)

A GLP-1 (7-36, NH₂) analog peptide containing a D-Ala substitution in the second residue as previously described (Siegel, et al. 1999 Regulatory Peptides 79:93-102) was synthesized and activated (Peptide 1).

The peptide was prepared on an ABI 433A Peptide Synthesizer using SynthAssist 2.0 Version for Fmoc/HBTU chemistry by the Fastmoc 0.25 mM Monitoring Previous Peak software. Universal PEG NovaTag resin (549 mg, 252 mmol) was used in the synthesis. Fmoc-Phe-Thr(YMe, MePro)-OH was used for the sixth and seventh amino acid position in the sequence. Fmoc-Ser(But)-Ser(YMe, MePro)-OH was used for the eleventh and twelfth amino acid position in the sequence. The final weight of the resin was 1.18 g.

The resin was washed 3×2 min with ethanol and 3×2 min with methylene chloride. To remove the pendant Mmt group, HOBt-hydrate (9.186 gm, 0.6 M) was dissolved in 100 mL methylene chloride/2,2,2-trifluoroethanol and 25 mL added to the resin and gently mixed for one hour. The solvent was removed by filtration and the sequence repeated three times. The resin was washed 3×2 min with methylene chloride, 1×2 min with methylene chloride in N-methylpyrrolidone and 3×2 min with N-methylpyrrolidone. To the resin was added tri-Boc-hydrazinoacetic acid (911.0 mg, 2.33 mM), HBTU (884.3 mg, 2.38 mM), HOBt/N-methylpyrrolidone (2.33 mL, 1 M) and 2.3 mL N-methylpyrrolidone and mixed well until all components dissolved. 4-Methylmorpholine (0.769 mL, 3 mM) was added, the pH checked by moistened paper strip (pH 8.5) and stirred for 19 hours at ambient temperature.

The resin was then washed 3×2 min with N-methylpyrrolidone, 1×2 min with methylene chloride/N-methylpyrrolidone, 3×2 min with methylene chloride, 3×2 min with methanol, 1×2 min with ethyl ether and dried under reduced pressure for two hours. The weight of the resin was 1.14 g.

The peptide was cleaved from the resin (0.371 g) by stirring in a scintillation vial using 20 mL of a cleavage mixture of trifluoroacetic acid (30 mL), phenol (2.25 g), dithiothrietol (1.5 g), thioanisole (1.5 mL), triisopropylsilane (1.5 mL), and water (1.5 mL) for two hours at ambient temperature. The resin was removed by filtration and the peptide was precipitated by the addition of precooled ethyl ether (600 mL). The resulting solid was isolated by filtration and washed with ethyl ether. The crude peptide was dried under reduced pressure to give 535 mg

The crude peptide was purified on two Vydac C-18 columns (10 mm, 2.5×25 cm), using a gradient of 0-40% (80% acetonitrile/0.1% trifluoroacetic acid in water) over 5 min and eluting on a gradient 40-60% (80% acetonitrile/0.1% trifluoroacetic acid in water) over 60 min at a flow rate of 6 mL/min. Fractions were collected, analyzed by HPLC and the pure fractions pooled and lyophilized to give 54.0 mg of white product. Capillary electrophoresis indicated a peak area of greater than 93%. (Molecular weight: Calcd:: 3,630.1; Monoisotopic MW: 3,627.9) Found: LC-MS: 3,630.8 Da [M+H]+SELDI-MS: 3,627.5 Da [M+H]+3,724/8 Da [M+97]+

### Example 1B

### (His-[D-Ala]-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-NH—CH2-CH2-(O—CH₂—CH₂)₁₂—CO-Gly-NH—NH2)

The GLP-1 (7-36, NH2) analog peptide containing a 2 D-Ala as above was used to prepare an alternatively activated reagent, Peptide 2.

The peptide was prepared on an ABI 433A Peptide Synthesizer using SynthAssist 2.0 Version for Fmoc/HBTU chemistry by the Fastmoc 0.1 mM Monitoring Previous Peak software. Fmoc-Gly-SASRIN resin (139 mg, 110 mmol) was used in the synthesis. Fmoc-Phe-Thr(YMe,MePro)-OH was used for the sixth and seventh amino acid position in the sequence. Fmoc-Ser(But)-Ser(YMe,Mepro)-OH was used for the eleventh and twelfth amino acid position in the sequence.

For the linking moiety, O—(N-Fmoc-2-aminoethyl)-0′-(2-carboxyethyl)-undecaethyleneglycol was used in the thirty-second amino acid position in the sequence The resin was washed with ethanol and dried overnight under reduced pressure. The final weight of the resin was 0.480 g.

The resin (189 mg) was mixed with 5 mL of 10% hydrazine (anhydrous) in dimethylformamide and stirred over 2 hrs at ambient temperature. The resin was filtered off, washed with 1 mL dimethylformamide and 100 mL of hot (70° C.) water was added to the filtrate. The filtrate cooled to ambient temperature for 1 hr and then refrigerated at 10° C. for 2 hrs. The white precipitate was filtered, washed with water (3×20 mL) and ethyl ether (3×40 mL) and then dried under reduced pressure to give 126 mg of a white solid. The protected peptide (120 mg) was deprotected using a 15 mL cleavage mixture of trifluoroacetic acid (20 mL), phenol (1.5 g), dithiothreitol (1.0 g), thioanisole (1.0 mL), TIS (1.0 mL), and water (1.0 mL) for two hours at ambient temperature. The resin was removed by filtration and the peptide was precipitated by the addition of precooled ethyl ether (400 mL), isolated by filtration and washed with ethyl ether. The crude peptide was dried under reduced pressure to give 95 mg of white solid.

The crude peptide was purified in two injections on two Vydac C-18 columns (10 mm, 2.5×25 cm), using a gradient of 0-30% (80% acetonitrile/0.1% trifluoroacetic acid in water) over 5 min and eluting on a gradient 30-60% (80% acetonitrile/0.1% trifluoroacetic acid in water) over 60 min at a flow rate of 6 mL/min. Fractions were collected, analyzed by HPLC and the pure fractions pooled and lyophilized to give 23 mg of white product. Capillary electrophoresis indicated a peak area of greater than 94%. (Molecular Weight: Calcd:: 4,026.5; Monoisotopic 4,024.1). Found: LC-MS: 4,028.0 Da [M+H]+.

### Example 1C

### (NH₂—NH—CH₂—CO—NH—CH₂—CH₂—O—(CH₂—CH₂—O)₁₀—CH₂—CH₂—O—CH₂—CH₂—CO—His-[D-Ala]-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-NH2)

The GLP-1 (7-36, NH2) analog peptide containing a 2 D-Ala as above was used to prepare an alternatively activated reagent, Peptide 3.

The peptide was prepared on an ABI 433A Peptide Synthesizer using SynthAssist 2.0 Version for Fmoc/HBTU chemistry by the Fastmoc 0.25 mM Monitoring Previous Peak software. Rink resin (833 mg, 250 mmol) was used in the synthesis. Fmoc-Gly-Thr(YMe, MePro)-OH, Fmoc-Phe-Thr(YMe, MePro)-OH and Fmoc-Val-Ser(YMe, MePro)-OH were used for the 21st, 23rd and 24th positions respectively. O—(N-Fmoc-2-aminoethyl)-0′-(2-carboxyethyl)-undecaethyleneglycol was used in the 28th position and tri-Boc-hydrazinoacetic acid was used in the 29th position. The final weight of the resin was 1.74 g. The peptide was simultaneously deprotected and removed from the resin with a cocktail of 1.5 g phenol, 3 ml ethanedithiol, 0.5 ml thioanisole, 0.5 ml water and 10 ml TFA for 4 hr at ambient temperature. The resin was removed by filtration and the peptide precipitated by the addition of diethyl ether. The solid was isolated by centrifugation, washing well with ether and drying under reduced pressure.

The material was purified using two Vydac C-18 columns (10 mm, 2.5×25 cm), using a gradient of 40-90% (80% acetonitrile/0.1% trifluoroacetic acid in water) over 90 min at a flow rate of 6 mL/min. Fractions were collected, analyzed by HPLC and the pure fractions pooled and lyophilized to give the peptide as a white power. Molecular Weight: Calcd. 4028.5, Monoisotopic 4026.1. Found: 4027.2 [M+H]⁺.

### Example 1D

### Preparation of a GLP-1 (7-36) Peptide-Linker-Hydrazide (NH₂—NH—CH₂—CO-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-NH₂)

The peptide is prepared on an ABI 433A Peptide Synthesizer using SynthAssist 2.0 Version for Fmoc/HBTU chemistry by the Fastmoc 0.25 mM Monitoring Previous Peak software. Rink resin is used in the synthesis. Fmoc-Gly-Thr(ΨMe, MePro)-OH, Fmoc-Phe-Thr(ΨMe, MePro)-OH and Fmoc-Val-Ser(ΨMe, MePro)-OH were used for the Gly-Thr, Phe-Thr and Val-Ser sequences respectively.

After the addition of His¹ of the GLP-1 sequence, Boc₃-hydrazinoacetic acid is coupled to the resin peptide. The peptide is simultaneously deprotected and removed from the resin with a cocktail of 1.5 g phenol, 3 ml ethanedithiol, 0.5 ml thioanisole, 0.5 ml water and 10 ml TFA for 4 hr at ambient temperature. The resin is removed by filtration and the peptide precipitated by the addition of diethyl ether. The solid is isolated by centrifugation, washing well with ether and drying under reduced pressure.

The material is purified using two Vydac C-18 columns (10 mm, 2.5×25 cm), using a gradient of 40-90% of 80% acetonitrile in 0.1% aqueous trifluoroacetic acid over 90 min at a flow rate of 6 mL/min. Fractions were collected, analyzed by HPLC and the pure fractions pooled and lyophilized to give the desired peptide hydrazine as a white power.

### Example 1E

### Preparation of a GLP-1 (7-36) Peptide-Linker-Hydrazino (NH₂—NH—CH₂—CO—NH—CH₂—CH₂—(O—CH₂—CH₂)₁₂—CO-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-NH₂)

An ABI 433 synthesizer is used with FastMoc chemistry (0.25 mmol scale). The pseudo-proline dipeptides Fmoc-Gly-Thr(ΨMe, MePro)-OH, Fmoc-Phe-Thr(ΨMe, MePro)-OH and Fmoc-Val-Ser(ΨMe, MePro)-OH were used for the dipeptides Gly-Thr, Phe-Thr and Val-Ser respectively. The protecting groups used were N-terminal Fmoc, His(Trt), Glu(O-t-butyl), Ser(O-t-butyl), Asp(O-t-butyl), Tyr(O-t-butyl), Gln(Trt), Lys(BOC) and Trp(BOC). 0.20 g of 0.53 meq/g Rink Amide ChemMatrix resinc was used in the synthesis. After the coupling of His¹ to the resin, O—(N-Fmoc-2-aminoethyl)-0′-(2-carboxyethyl)-undecaethyleneglycol (Fmoc-PEG1₂—CO₂H) is coupled to the peptide resin, followed by the coupling of Boc₃-hydrazinoacetic acid. The final weight of peptide resin is 0.677 g.

The peptide is simultaneously deprotected and cleaved from the resin using a mixture of 10 ml TFA, 3 ml ethanedithiol, 1.5 g phenol, 0.5 ml water and 0.5 ml of thioanisole for 4 hr at ambient temperature. The resin is removed by filtration and the filtrate run directly into 250 ml of cold diethyl ether. The resulting solid is isolated by centrifugation, washed with diethyl ether by suspending in ether and centrifuging, and dried under reduced pressure to give 400 mg of a white solid.

The crude peptide is injected in aliquots onto two Vydac C-18 columns (4.6×250 mm, 10 m) in tandem and eluted with a linear gradient of 30-60% of 80% acetonitrile in 0.1% aqueous TFA over 90 min at a flow rate of 5 ml/min. The column is monitored at 214 nm. Fractions were analyzed and those containing the correct product were pooled and lyophilized to give the desired product as a white solid. (Molecular Weight: Calcd. for C₁₈₀H₂₈₆N₄₄O₆₀: 4026.55. Found: 4,026.90)

### Example 1F

### Preparation of a GLP-1 (7-36) Peptide-Linker (NH₂—NH—CO—CH₂—CH₂—CO-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-NH₂)

An ABI 433 synthesizer is used with FastMoc chemistry (0.25 mmol scale). The pseudo-proline dipeptides Fmoc-Gly-Thr(ΨMe, MePro)-OH, Fmoc-Phe-Thr(ΨMe, MePro)-OH and Fmoc-Val-Ser(ΨMe, MePro)-OH were used for the dipeptides Gly-Thr, Phe-Thr and Val-Ser respectively. The protecting groups used were N-terminal Fmoc, His(Trt), Glu(O-t-butyl), Ser(O-t-butyl), Asp(O-t-butyl), Tyr(O-t-butyl), Gln(Trt), Lys(BOC) and Trp(BOC). 0.20 g of 0.53 meq/g Rink Amide ChemMatrix resinc was used in the synthesis. After the coupling of His¹ to the resin, butanedioic acid monomethyl ester is coupled to the peptide resin. The resulting peptide resin is washed with N-methylpyrrolidone and ethanol and dried under reduced pressure to a constant weight.

The peptide resin is mixed with 5 mL of 10% hydrazine (anhydrous) in dimethylformamide and stirred over 2 hrs at ambient temperature. The resin is filtered off, washed with 1 mL dimethylformamide and 100 mL of hot (70° C.) water is added to the filtrate. The filtrate is refrigerated at 4° C. for 24 hrs. The resulting precipitate is filtered, washed with water (3×20 mL) and ethyl ether (3×40 mL) and then dried under reduced pressure to give a white solid. The protected peptide is deprotected using a 15 mL cleavage mixture of trifluoroacetic acid (20 mL), phenol (1.5 g), dithiothreitol (1.0 g), thioanisole (1.0 mL), triisopropylsilane (1.0 mL), and water (1.0 mL) for two hours at ambient temperature. The resin is removed by filtration and the peptide is precipitated by the addition of cold ethyl ether (400 mL), isolated by filtration and washed with ethyl ether and dried under reduced pressure.

The crude peptide is injected in aliquots onto two Vydac C-18 columns (4.6×250 mm, 10 m) in tandem and eluted with a linear gradient of 20-70% of 80% acetonitrile in 0.1% aqueous TFA over 90 min at a flow rate of 5 ml/min. The column is monitored at 214 nm. Fractions were analyzed and those containing the correct product were pooled and lyophilized to give the desired peptide hydrazide as a white solid.

### Example 1G

### Preparation of a GLP-1 (7-36) Peptide-Linker-Hydrazine (NH₂—NH—CO—CH₂—CH₂—CO—NH—CH₂—CH₂—(O—CH₂—CH₂)₁₂—CO-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-NH₂)

An ABI 433 synthesizer is used with FastMoc chemistry (0.25 mmol scale). The pseudo-proline dipeptides Fmoc-Gly-Thr(ΨMe, MePro)-OH, Fmoc-Phe-Thr(ΨMe, MePro)-OH and Fmoc-Val-Ser(ΨMe, MePro)-OH were used for the dipeptides Gly-Thr, Phe-Thr and Val-Ser respectively. The protecting groups used were N-terminal Fmoc, His(Trt), Glu(O-t-butyl), Ser(O-t-butyl), Asp(O-t-butyl), Tyr(O-t-butyl), Gln(Trt), Lys(BOC) and Trp(BOC). 0.175 g of 0.53 meq/g Rink Amide ChemMatrix resin is used in the synthesis. After the coupling of His¹ to the resin, O—(N-Fmoc-2-aminoethyl)-O′-(2-carboxyethyl)-undecaethyleneglycol (Fmoc-PEG₁₂-CO₂H) is coupled to the peptide resin, followed by the coupling of butanedioic acid, monomethyl ester.

The resulting peptide resin is mixed with 5 mL of 10% hydrazine (anhydrous) in dimethylformamide and stirred over 2 hrs at ambient temperature. The resin is filtered off, washed with 1 mL dimethylformamide and 100 mL of hot (70° C.) water is added to the filtrate. The filtrate is refrigerated at 4° C. overnight. The resulting precipitate is filtered, washed with water (3×20 mL) and ethyl ether (3×40 mL) and then dried under reduced pressure to give a white solid. The protecting groups were removed from the peptide using a 15 mL cleavage mixture of trifluoroacetic acid (20 mL), phenol (1.5 g), dithiothreitol (1.0 g), thioanisole (1.0 mL), triisopropylsilane (1.0 mL), and water (1.0 mL) for two hours at ambient temperature. The resin is removed by filtration and the peptide is precipitated by the addition of precooled ethyl ether (400 mL), isolated by filtration and washed with ethyl ether and dried under reduced pressure.

The crude peptide is injected in aliquots onto two Vydac C-18 columns (4.6×250 mm, 10 m) in tandem and eluted with a linear gradient of 20-70% of 80% acetonitrile in 0.1% aqueous TFA over 90 min at a flow rate of 5 ml/min. The column is monitored at 214 nm. Fractions were analyzed and those containing the correct product were pooled and lyophilized to give the desired peptide hydrazide as a white solid.

### Example 1H

### Preparation of a GLP-1 (7-36) Peptide-Hydrazine (His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-NH—NH₂)

The peptide is prepared on an ABI 433A Peptide Synthesizer using SynthAssist 2.0 Version for Fmoc/HBTU chemistry by the Fastmoc 0.25 mM Monitoring Previous Peak software. Rink resin is used in the synthesis. Fmoc-Gly-Thr(ΨMe, MePro)-OH, Fmoc-Phe-Thr(ΨMe, MePro)-OH and Fmoc-Val-Ser(ΨMe, MePro)-OH were used for the Gly-Thr, Phe-Thr and Val-Ser respectively.

The resulting peptide resin is mixed with 5 mL of 10% hydrazine

(anhydrous) in dimethylformamide and stirred over 2 hrs at ambient temperature. The resin is filtered off, washed with 1 mL dimethylformamide and 125 mL of hot (70° C.) water is added to the filtrate. The filtrate is refrigerated at 4° C. overnight. The resulting precipitate is filtered, washed with water (3×20 mL) and ethyl ether (3×40 mL) and then dried under reduced pressure to give a white solid. The protected peptide is deprotected using a 15 mL cleavage mixture of trifluoroacetic acid (20 mL), phenol (1.5 g), dithiothreitol (1.0 g), thioanisole (1.0 mL), triisopropylsilane (1.0 mL), and water (1.0 mL) for two hours at ambient temperature. The resin is removed by filtration and the peptide is precipitated by the addition of precooled ethyl ether (400 mL), isolated by filtration and washed with ethyl ether and dried under reduced pressure.

The crude peptide is injected in aliquots onto two Vydac C-18 columns (4.6×250 mm, 10 m) in tandem and eluted with a linear gradient of 20-70% of 80% acetonitrile in 0.1% aqueous TFA over 90 min at a flow rate of 5 ml/min. The column is monitored at 214 nm. Fractions were analyzed and those containing the correct product were pooled and lyophilized to give the desired peptide hydrazide as a white solid.

### Example 1I

### Preparation of a GLP-1 (7-36) Peptide Derivative-Linker-Hydrazine (His-[D-Ala]-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-NH—CH₂—CH₂—(O—CH₂—CH₂)₁₂—CO-Gly-NH—NH₂)

The peptide is prepared on an ABI 433A Peptide Synthesizer using SynthAssist 2.0 Version for Fmoc/HBTU chemistry by the Fastmoc 0.1 mM Monitoring Previous Peak software. Fmoc-Gly-SASRIN resin is used in the synthesis. Fmoc-Phe-Thr(ΨMe, MePro)-OH and Fmoc-Ser(t-Bu)-Ser(ΨMe, MePro)-OH were used for the Phe-Thr and Ser-Ser sequences respectively.

O—(N-Fmoc-2-aminoethyl)-0′-(2-carboxyethyl)-undecaethyleneglycol (Fmoc-PEG₁₂-CO₂H) is coupled to the resin, followed by the coupling of the individual amino acids to give the protected peptide-linker-resin. The resin is washed with ethanol and dried overnight under reduced pressure.

The peptide resin is mixed with 5 mL of 10% hydrazine (anhydrous) in dimethylformamide and stirred over 2 hrs at ambient temperature. The resin is filtered off, washed with 1 mL dimethylformamide and 100 mL of hot (70° C.) water is added to the filtrate. The filtrate cooled to ambient temperature for 1 hr and then refrigerated at 4° C. for 22 hrs. The white precipitate is filtered, washed with water (3×20 mL) and ethyl ether (3×40 mL) and then dried under reduced pressure to give a white solid. The protected peptide is deprotected using a 15 mL cleavage mixture of trifluoroacetic acid (20 mL), phenol (1.5 g), dithiothreitol (1.0 g), thioanisole (1.0 mL), triisopropylsilane (1.0 mL), and water (1.0 mL) for two hours at ambient temperature. The resin is removed by filtration and the peptide is precipitated by the addition of precooled ethyl ether (400 mL), isolated by filtration and washed with ethyl ether. The crude peptide is dried under reduced pressure.

The crude peptide is purified in aliquots on two Vydac C-18 columns (10 mm, 2.5×25 cm), using a gradient of 30-70% of 80% acetonitrile in 0.1% trifluoroacetic acid in water over 60 min at a flow rate of 6 mL/min. Fractions were collected, analyzed by HPLC and the pure fractions pooled and lyophilized to give 23 mg of white product. Capillary electrophoresis indicated a peak area of greater than 94%. (Molecular Weight: Calcd:: 4,026.5; Monoisotopic 4,024.1). Found: LC-MS: 4,028.0 Da [M+H]⁺.

### Example 1J

### Preparation of a GLP-1 (7-36) Peptide Derivative-Linker-Hydrazine (His-[D-Ala]-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-PEG₃-CO—CH₂—NH—NH₂)

The peptide is prepared on an ABI 433A Peptide Synthesizer using SynthAssist 2.0 Version for Fmoc/HBTU chemistry by the Fastmoc 0.25 mM Monitoring Previous Peak software. Universal PEG NovaTag resin (549 mg, 252 mmol) is used in the synthesis. Fmoc-Phe-Thr(ΨMe, MePro)-OH and Fmoc-Ser(But)-Ser(ΨMe, MePro)-OH were used for the Phe-The and Ser-Ser sequences respectively. The final weight of the resin is 1.18 g.

The resin is washed 3×2 min with ethanol and 3×2 min with methylene chloride. To remove the Mmt group, HOBt-hydrate (9.186 gm, 0.6 M) is dissolved in 100 mL methylene chloride/2,2,2-trifluoroethanol and 25 mL added to the resin and gently mixed for one hour. The solvent is removed by filtration and the sequence repeated three times. The resin is washed 3×2 min with methylene chloride, 1×2 min with methylene chloride in N-methylpyrrolidone and 3×2 min with N-methylpyrrolidone. To the resin is added Boc₃-hydrazinoacetic acid (911.0 mg, 2.33 mM), HBTU (884.3 mg, 2.38 mM), HOBt in N-methylpyrrolidone (2.33 mL, 1 M) and 2.3 mL N-methylpyrrolidone and mixed well until all components dissolved. N-methylmorpholine (0.769 mL, 3 mM) is added, the pH checked by moistened paper strip (pH 8.5) and stirred for 19 hours at ambient temperature.

The resin is then washed 3×2 min with N-methylpyrrolidone, 1×2 min with methylene chloride/N-methylpyrrolidone, 3×2 min with methylene chloride, 3×2 min with methanol, 1×2 min with ethyl ether and dried under reduced pressure for two hours. The weight of the resin is 1.14 g.

The peptide is cleaved from the resin (0.371 g) by stirring in a scintillation vial using 20 mL of a cleavage mixture of trifluoroacetic acid (30 mL), phenol (2.25 g), dithiothrietol (1.5 g), thioanisole (1.5 mL), triisopropylsilane (1.5 mL), and water (1.5 mL) for two hours at ambient temperature. The resin is removed by filtration and the peptide is precipitated by the addition of precooled ethyl ether (600 mL). The resulting solid is isolated by filtration and washed with ethyl ether. The crude peptide is dried under reduced pressure to give 535 mg

The crude peptide is purified on two Vydac C-18 columns (10 mm, 2.5×25 cm), using a gradient of 40-60% of 80% acetonitrile in 0.1% trifluoroacetic acid in water over 60 min at a flow rate of 6 mL/min. Fractions were collected, analyzed by HPLC and the pure fractions pooled and lyophilized to give 54.0 mg of white product. Capillary electrophoresis indicated a peak area of greater than 93%. (Molecular weight: Calcd: 3,630.1; Monoisotopic MW: 3,627.9) Found: LC-MS: 3,630.8 Da [M+H]+SELDI-MS: 3,627.5 Da [M+H]+3,724/8 Da [M+97]+

### Example 1K

### Preparation of a GLP-1 (7-36) Peptide-Linker Hydrazine (His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-PEG₃-CO—(CH₂—CH₂—O)₁₂—CH₂—CH₂—NH—CO—CH₂—CH₂—CO—NH—NH₂)

The peptide is prepared on an ABI 433A Peptide Synthesizer using SynthAssist 2.0 Version for Fmoc/HBTU chemistry by the Fastmoc 0.25 mM Monitoring Previous Peak software. Universal PEG NovaTag resin (549 mg, 252 mmol) is used in the synthesis. Fmoc-Phe-Thr(ΨMe, MePro)-OH and Fmoc-Ser(But)-Ser(ΨMe, MePro)-OH were used for the Phe-The and Ser-Ser sequences respectively. The final weight of the resin is 1.18 g.

The resin is washed 3×2 min with ethanol and 3×2 min with methylene chloride. To remove the Mmt group, HOBt-hydrate (9.186 gm, 0.6 M) is dissolved in 100 mL methylene chloride/2,2,2-trifluoroethanol and 25 mL added to the resin and gently mixed for one hour. The solvent is removed by filtration and the sequence repeated three times. The resin is washed 3×2 min with methylene chloride, 1×2 min with methylene chloride in N-methylpyrrolidone and 3×2 min with N-methylpyrrolidone. To the free amino group of the peptide resin is added O—(N-Fmoc-2-aminoethyl)-0′-(2-carboxyethyl)-undecaethyleneglycol (Fmoc-PEG₁₂-CO₂H) followed by the addition of butanedioic acid monomethyl ester using HBTU and HOBt in N-methylpyrrolidone

The resin is then washed 3×2 min with N-methylpyrrolidone, 1×2 min with methylene chloride/N-methylpyrrolidone, 3×2 min with methylene chloride, 3×2 min with methanol, 1×2 min with ethyl ether and dried under reduced pressure for two hours.

The peptide resin is mixed with 25 mL of 10% hydrazine (anhydrous) in dimethylformamide and stirred over 2 hrs at ambient temperature. The resin is filtered off, washed with 1 mL dimethylformamide and 250 mL of hot (70° C.) water is added to the filtrate. The filtrate cooled to ambient temperature for 1 hr and then refrigerated at 4° C. for 16 hrs. The white precipitate is filtered, washed with water (3×20 mL) and ethyl ether (3×40 mL) and then dried under reduced pressure to give a white solid. The protected peptide is deprotected using a cleavage mixture of trifluoroacetic acid (20 mL), phenol (1.5 g), dithiothreitol (1.0 g), thioanisole (1.0 mL), triisopropylsilane (1.0 mL), and water (1.0 mL) for two hours at ambient temperature. The resin is removed by filtration and the peptide is precipitated by the addition of precooled ethyl ether (400 mL), isolated by filtration and washed with ethyl ether. The crude peptide is dried under reduced pressure.

The crude peptide is purified on two Vydac C-18 columns (10 mm, 2.5×25 cm), using a gradient of 40-80% of 80% acetonitrile in 0.1% trifluoroacetic acid in water over 60 min at a flow rate of 6 mL/min. Fractions were collected, analyzed by HPLC and the pure fractions pooled and lyophilized to give the desired peptide derivative.

### Example 1L

### Preparation of a GLP-1 (7-36) Peptide-Linker-Hydrazine (His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-PEG₃-CO—(CH₂—CH₂—O)₁₂—CH₂—CH₂—NH—CO—CH₂—NH—NH₂)

The peptide is prepared on an ABI 433A Peptide Synthesizer using SynthAssist 2.0 Version for Fmoc/HBTU chemistry by the Fastmoc 0.25 mM Monitoring Previous Peak software. Universal PEG NovaTag resin is used in the synthesis. Fmoc-Phe-Thr(ΨMe, MePro)-OH and Fmoc-Ser(But)-Ser(ΨMe, MePro)-OH were used for the Phe-The and Ser-Ser sequences respectively.

The resin is washed 3×2 min with ethanol and 3×2 min with methylene chloride. To remove the Mmt group, HOBt-hydrate (9.186 gm, 0.6 M) is dissolved in 100 mL methylene chloride/2,2,2-trifluoroethanol and 25 mL added to the resin and gently mixed for one hour. The solvent is removed by filtration and the sequence repeated three times. The resin is washed 3×2 min with methylene chloride, 1×2 min with methylene chloride in N-methylpyrrolidone and 3×2 min with N-methylpyrrolidone. To the free amino group of the peptide resin is added O—(N-Fmoc-2-aminoethyl)-0′-(2-carboxyethyl)-undecaethyleneglycol (Fmoc-PEG₁₂-CO₂H) followed by the addition of Boc₃-hydrazinoacetic acid using HBTU and HOBt in N-methylpyrrolidone

The resin is then washed 3×2 min with N-methylpyrrolidone, 1×2 min with methylene chloride/N-methylpyrrolidone, 3×2 min with methylene chloride, 3×2 min with methanol, 1×2 min with ethyl ether and dried under reduced pressure for two hours.

The peptide is cleaved from the resin using a cleavage mixture of trifluoroacetic acid (30 mL), phenol (2.25 g), dithiothrietol (1.5 g), thioanisole (1.5 mL), triisopropylsilane (1.5 mL), and water (1.5 mL) for two hours at ambient temperature. The resin is removed by filtration and the peptide is precipitated by the addition of precooled ethyl ether (600 mL). The resulting solid is isolated by filtration and washed with ethyl ether. The crude peptide is dried under reduced pressure to give a white solid.

The crude peptide is purified in aliquots on two Vydac C-18 columns (10 mm, 2.5×25 cm), using a gradient of 20-70% of 80% acetonitrile in 0.1% trifluoroacetic acid in water over 60 min at a flow rate of 6 mL/min. Fractions were collected, analyzed by HPLC and the pure fractions pooled and lyophilized to give the desired peptide derivative.

### Example 1M

### Preparation of a Amidated GLP-1 (7-36) Peptide-Linker Hydrazine (NH₂—NH—CH₂—CH₂—(O—CH₂—CH₂)₁₂—CO-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-NH₂)

An ABI 433 synthesizer is used with FastMoc chemistry (0.25 mmol scale). The pseudo-proline dipeptides Fmoc-Gly-Thr(ΨMe, MePro)-OH, Fmoc-Phe-Thr(ΨMe, MePro)-OH and Fmoc-Val-Ser(ΨMe, MePro)-OH were used for the dipeptides Gly-Thr, Phe-Thr and Val-Ser respectively. The protecting groups used were N-terminal Fmoc, His(Trt), Glu(O-t-butyl), Ser(O-t-butyl), Asp(O-t-butyl), Tyr(O-t-butyl), Gln(Trt), Lys(BOC) and Trp(BOC). 0.20 g of 0.53 meq/g Rink Amide ChemMatrix resinc was used in the synthesis. After the coupling of His¹ to the resin, O-Boc₃-2-hydrazinoethyl)-0′-(2-carboxyethyl)-undecaethyleneglycol (Boc₃-hydrazino-PEG₁₂-CO₂H) is coupled to the peptide resin.

The peptide is simultaneously deprotected and cleaved from the resin using a mixture of 10 ml TFA, 3 ml ethanedithiol, 1.5 g phenol, 0.5 ml water and 0.5 ml of thioanisole for 4 hr at ambient temperature. The resin is removed by filtration and the filtrate run directly into 250 ml of cold diethyl ether. The resulting solid is isolated by centrifugation, washed with diethyl ether by suspending in ether and centrifuging, and dried under reduced pressure to give 400 mg of a white solid.

The crude peptide is injected in aliquots onto two Vydac C-18 columns (4.6×250 mm, 10 m) in tandem and eluted with a linear gradient of 20-70% of 80% acetonitrile in 0.1% aqueous TFA over 90 min at a flow rate of 5 ml/min. The column is monitored at 214 nm. Fractions were analyzed and those containing the correct product were pooled and lyophilized to give the desired product as a white solid.

### Example 1N

### Preparation of a GLP-1 (7-36) Peptide-Linker Hydrazine (His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-PEG₃-CO—(CH₂—CH₂—O)₁₂—CH₂—CH₂—NH—NH₂)

The peptide is prepared on an ABI 433A Peptide Synthesizer using SynthAssist 2.0 Version for Fmoc/HBTU chemistry by the Fastmoc 0.25 mM Monitoring Previous Peak software. Universal PEG NovaTag resin is used in the synthesis. Fmoc-Phe-Thr(ΨMe, MePro)-OH and Fmoc-Ser(But)-Ser(ΨMe, MePro)-OH were used for the Phe-The and Ser-Ser sequences respectively.

The resin is washed 3×2 min with ethanol and 3×2 min with methylene chloride. To remove the Mmt group, HOBt-hydrate is dissolved in 100 mL methylene chloride/2,2,2-trifluoroethanol and 25 mL added to the resin and gently mixed for one hour. The solvent is removed by filtration and the sequence repeated three times. The resin is washed 3×2 min with methylene chloride, 1×2 min with methylene chloride in N-methylpyrrolidone and 3×2 min with N-methylpyrrolidone. To the free amino group of the peptide resin is added O-(Boc₃-2-hydrazinoethyl)-0′-(2-carboxyethyl)-undecaethyleneglycol (Boc₃-hydrazino-PEG₁₂-CO₂H) using HBTU and HOBt in N-methylpyrrolidone

The resin is then washed 3×2 min with N-methylpyrrolidone, 1×2 min with methylene chloride/N-methylpyrrolidone, 3×2 min with methylene chloride, 3×2 min with methanol, 1×2 min with ethyl ether and dried under reduced pressure for two hours.

The peptide is cleaved from the resin using a cleavage mixture of trifluoroacetic acid (30 mL), phenol (2.5 g), dithiothrietol (1.5 g), thioanisole (1.5 mL), triisopropylsilane (1.5 mL), and water (1.5 mL) for two hours at ambient temperature. The resin is removed by filtration and the peptide is precipitated by the addition of precooled ethyl ether (600 mL). The resulting solid is isolated by filtration and washed with ethyl ether. The crude peptide is dried under reduced pressure to give a white solid.

The crude peptide is purified in aliquots on two Vydac C-18 columns (10 mm, 2.5×25 cm), using a gradient of 20-70% of 80% acetonitrile in 0.1% trifluoroacetic acid in water over 60 min at a flow rate of 6 mL/min. Fractions were collected, analyzed by HPLC. Pure fractions pooled and lyophilized to give the desired peptide derivative.

### Example 1O

### Preparation of an amidated GLP-1 (7-36) Peptide-Linker-Hydrazine (NH₂—NH—CH₂—CO—NH—CH₂—CH₂—CH₂—(O—CH₂—CH₂)₂—O—CH₂—CH₂—CH₂—NH—CH₂—O—CH₂—CO-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg-Gly-NH₂)

An ABI 433 synthesizer is used with FastMoc chemistry (0.25 mmol scale). The pseudo-proline dipeptides Fmoc-Gly-Thr(ΨMe, MePro)-OH, Fmoc-Phe-Thr(ΨMe, MePro)-OH and Fmoc-Val-Ser(ΨMe, MePro)-OH were used for the dipeptides Gly-Thr, Phe-Thr and Val-Ser respectively. The protecting groups used were N-terminal Fmoc, His(Trt), Glu(O-t-butyl), Ser(O-t-butyl), Asp(O-t-butyl), Tyr(O-t-butyl), Gln(Trt), Lys(BOC) and Trp(BOC). Rink Amide ChemMatrix resinc was used in the synthesis. After the coupling of His¹ to the resin, {3-[2-(2-{2-[3-(9H-Fluoren-9-yloxycarbonylamino)-propoxy]-ethoxy}-ethoxy)-ethylamino]-1-methyl-2-oxo-propoxy}-acetic acid (Fmoc-PEG₂-CO₂H) is coupled to the peptide resin, followed by the coupling of Boc₃-hydrazinoacetic acid.

The peptide is simultaneously deprotected and cleaved from the resin using a mixture of TFA, 3 ml ethanedithiol, 1.5 g phenol, 0.5 ml water and 0.5 ml of thioanisole for 4 hr at ambient temperature. The resin is removed by filtration and the filtrate run directly into 250 ml of cold diethyl ether. The resulting solid is isolated by centrifugation, washed with diethyl ether by suspending in ether and centrifuging, and dried under reduced pressure to give the crude peptide as a white solid.

The crude peptide is injected in aliquots onto two Vydac C-18 columns (4.6×250 mm, 10 m) in tandem and eluted with a linear gradient of 30-60% of 80% acetonitrile in 0.1% aqueous TFA over 90 min at a flow rate of 5 ml/min. The column is monitored at 214 nm. Fractions were analyzed and those containing the correct product were pooled and lyophilized to give the desired product as a white solid.

### Example 2

### Preparation of Glyoxylyl-FC

Human antibodies of the IgG1 class/subclass are cleavable by papain at a site on the heavy chain which produces an Fc fragment with an N-terminal threonine. In the present studies, an murine-human chimera comprising the human constant regions of the IgG4 antibody, 7E3, is used as the Fc, (Kohmura et al. 1993 Arterioscler Thromb. 13:1837-42; EP418316).

Deglycosylation of 7E3 IgG Fc

135 ml of Fc (5 mg/ml) is dialyzed into 10 mM Tris, pH 7.5. To the dialyzate is added 100 ml of PNGase F (500,000 u/ml) and the resulting solution incubated at 37° for 3 days. The deglycosylated Fc is purified on a TosoHaas phenyl 5PW column (5.5×200 mm, 10 m) eluted with the gradient of 0-50% B at a flow rate of 11 ml/min (Buffer A: 0.1 M sodium phosphate, 1 M ammonium sulfate, pH 6.5; Buffer B: 0.1 M sodium phosphate, pH 6.5. Molecular Weight: Calcd: 49,864.4. Found: 49,868.4.

### Oxidation of Deglycosylated Fc

55 ml of deglycosylated Fc (8.9 mg/ml from Experiment #205) is dialyzed into 1% NaHCO₃, pH 8.4, to give 56.3 ml of 8.6 mg/ml. This is adjust the concentration to 5.1 mg/ml (10-4 mmol of protein/ml, this is equivalent to 2×10-4 mmol of N-terminal threonine/ml) by the addition of 40.6 ml of 1% NaHCO₃, pH 8.4. A solution of 12.5 mg/ml of methionine in 1% NaHCO₃, pH 8.4 is prepared and 11.9 ml is added to the Fc solution.

A solution of 20 mg/ml of NaIO4 in water is prepared. 2.12 ml (42.4 mg) is added to the Fc. The reaction mixture is gently agitated at ambient temperature for 15 minutes. Ethylene glycol (2.8 g, 2.3 ml) is added and the reaction gently agitated for an additional 20 minutes. The solution is dialyzed into 0.1 M NaOAc, pH 4.5 to give 120 ml of 4.0 mg/ml. The solution is divided into 2.5 ml aliquots, frozen at −20° C. and used without further purification.

### Example 3

### Preparation of Peptide-FC Conjugate

### Using Peptide 1, Example 1A:

To glyoxylyl-Fc (2.5 ml, 4 mg/ml) is added 12 mg of the activated GLP-1 analog, Peptide 1A. The tube is placed in the refrigerator at 4° C. for 24 hours. A solution of 1 mg/ml solution of NaBH₃CN is prepared and 100 ml is added to the reaction and the reaction is returned to the refrigerator overnight. To the sample is added 100 mg of ammonium sulfate. The sample is injected onto a TosoHaas phenyl 5PW column (5.5×200 mm, 10 m) eluted with the gradient of 0-100% B at a flow rate of 11 ml/min (Buffer A: 0.1 M sodium phosphate, 1 M ammonium sulfate, pH 6.5; Buffer B: 0.1 M sodium phosphate, pH 6.5. Fractions were pooled, concentrated to ca. 8 ml and dialyzed into PBS. Molecular Weight: Calcd: 57,005.8, monoisotopic 56,970.4. Found: 57,004.3.

### Using Peptide 2, Example 1B:

To glyoxylyl-Fc (2.5 ml, 4 mg/ml) as added 10 mg of activated GLP-1 analog, Peptide 1B. The tube is placed in the refrigerator at 4° C. for 24 hours. A solution of 1 mg/ml solution of NaBH₃CN is prepared and 100 ml is added to the reaction and the reaction is returned to the refrigerator overnight. To the sample is added 100 mg of ammonium sulfate. The sample is injected onto a TosoHaas phenyl 5PW column (5.5×200 mm, 10 m) eluted with the gradient of 0-100% B at a flow rate of 11 ml/min (Buffer A: 0.1 M sodium phosphate, 1 M ammonium sulfate, pH 6.5; Buffer B: 0.1 M sodium phosphate, pH 6.5. Fractions were pooled, concentrated to ca. 8 ml and dialyzed into PBS. Molecular Weight: Calcd: 57,883.8, monoisotopic 57,850.9. Found: 57,796.5.

### Using Peptide 3, Example 1C.

To glyoxylyl-Fc (2.5 ml, 4 mg/ml) as added 8 mg of peptide 1C. The tube is placed in the refrigerator at 4° C. for 24 hours. A solution of 1 mg/ml solution of NaBH₃CN is prepared and 100 ml is added to the reaction and the reaction is returned to the refrigerator overnight. To the sample is added 100 mg of ammonium sulfate. The sample is injected onto a TosoHaas phenyl 5PW column (5.5×200 mm, 10 m) eluted with the gradient of 0-100% B at a flow rate of 11 ml/min (Buffer A: 0.1 M sodium phosphate, 1 M ammonium sulfate, pH 6.5; Buffer B: 0.1 M sodium phosphate, pH 6.5. Fractions were pooled, concentrated to ca. 8 ml and dialyzed into PBS. Molecular Weight: Calcd: 57,802.6, monoisotopic 57,766.8. Found: 57,811.1.

Monovalent Construct from Peptide 3:

To glyoxylaldehyde-Fc (2.5 ml, 4 mg/ml) as added 1.5 mg of peptide 3. The tube is placed in the refrigerator at 4° C. for 24 hours. A solution of 1 mg/ml solution of NaBH₃CN is prepared and 100 ml is added to the reaction and the reaction is returned to the refrigerator overnight. To the sample is added 100 mg of ammonium sulfate. The sample is injected onto a TosoHaas phenyl 5PW column (5.5×200 mm, 10 m) eluted with the gradient of 50-100% B at a flow rate of 11 ml/min (Buffer A: 0.1 M sodium phosphate, 1 M ammonium sulfate, pH 6.5; Buffer B: 0.1 M sodium phosphate, pH 6.5. Fractions were pooled, concentrated to ca. 8 ml and dialyzed into PBS. Molecular Weight: Calcd: 53,792.2, monoisotopic 53,758.5. Found: 53,803.7.

### Example 4

### Bioactivity of Peptide-FC Conjugates

The peptide-conjugates were tested for activity using the cAMP assay which measure cAMP produced upon modulation of adenylyl cyclase activity by GPCRs.

cAMP Assay The LANCE™ cAMP assay (Hemmila I. 1999. LANCE™: Homogeneous Assay Platform for HTS. J Biomol Screen. 4(6), 303-308) is a homogeneous time-resolved fluorescence resonance energy transfer (TR-FRET) immunoassay. The assay is based on the competition between a europium-labeled cAMP tracer and sample cAMP for binding sites on cAMP-specific antibodies labeled with the dye Alexa Fluor® 647. The europium-labeled tracer complex is formed by the tight interaction between Biotin-cAMP and streptavidin labeled with Europium-W8044 chelate. When antibodies are bound to the Eu-SA/b-cAMP tracer, light pulse 340 nm excites the Eu-chelate molecules of the tracer. The energy emitted by the Eu-chelate is transferred to an Alexa molecule on the antibodies, which in turn emits light at 665 nm. The fluorescence intensity measure at 665 nm will decrease in the presence of cAMP from the test samples and resulting signals will be inversely proportional to the cAMP concentration of a sample (LANCE cAMP manual).

Cells and Assay. INS-1E cells (from Claes Wollheim, Geneva, Switzerland. Endocrinology, 1992, 130(1):167-178) were cultured in RPMI 1640/10% FBS/1% L-glutamine/1% sodium pyruvate/1% Non-essential Amino Acids/50 μM beta-mercaptoethanol and maintained at 37° C. in a humidified incubator with 5% CO₂. Cells were passaged by trypsinization and sub-cultured every 7 days.

For the assay, INS-1E cells were plated at confluence in 96-well plates (Costar 3610) and allowed to recover for 4 days in normal growth media. Media was aspirated from the wells and 24 ul of Alexa Fluor® 647 anti-cAMP antibody (LANCE cAMP Kit, Perkin Elmer, Boston, Mass.) was added followed by 24 ul of a dilution series of test article (in PBS/0.5% BSA/0.5 mM IBMX). The cells were stimulated at room temperature for 7 minutes and then lysed in buffer containing the Eu-SA/b-cAMP tracer. The plates were incubated at room temperature for 1 hour and then fluorescence intensity was measured at 665 nm. Cyclic AMP concentrations were determined against a standard curve.

### Results

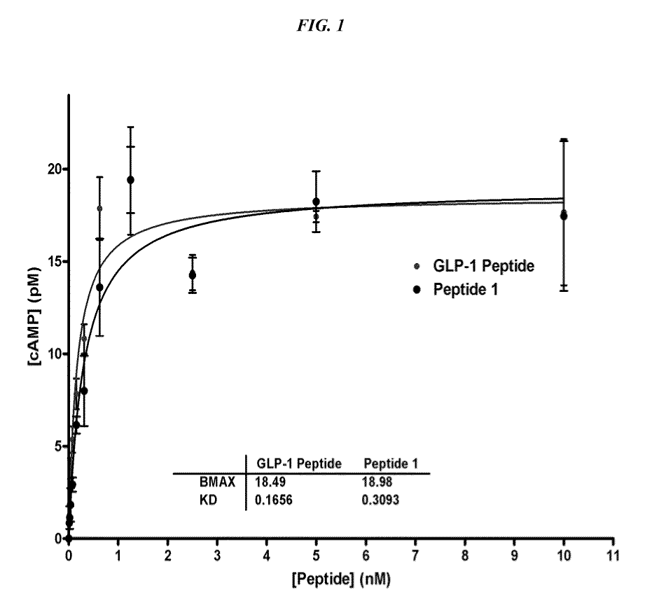

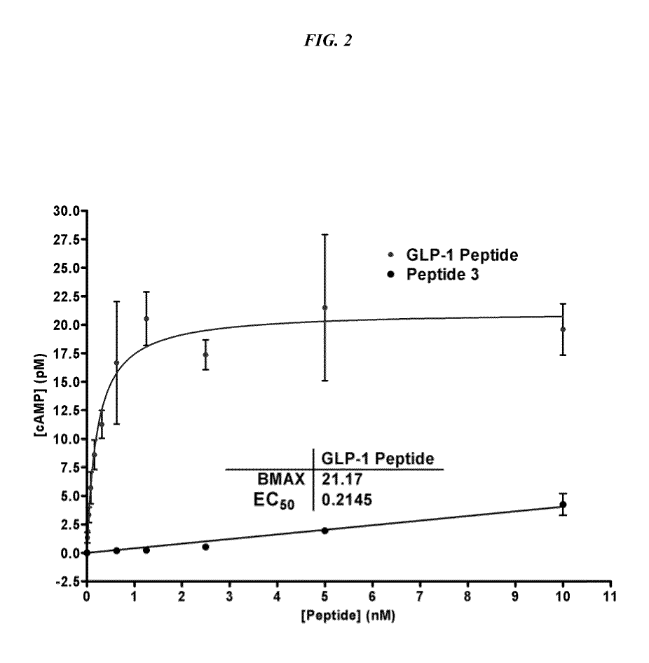

The ability of Peptide 1, as shown in FIG. 1 and as prepared in Example 1A, to stimulated cAMP in the INS-1E cells was compared to wild-type GLP-1. The results, shown graphically in FIG. 2, demonstrate that there was no loss in bioactivity of the modified peptide.

The ability of the modified GLP-1 peptides conjugated to human Fc-region as described in Example 3 to stimulate cAMP in INS-1E cells was compared as shown graphically in FIG. 4. As shown, all of the constructs exhibited activity. Activity of the N-terminally linked GLP-1 analog (Peptide 3) was unanticipated as N-terminal truncation of GLP-1 by 2 amino acids was previously shown to produce weak agonist activity, and 8-amino acid N-terminal truncation inactivated the peptide (Montrose-Rafizadeh, et al. 1997. J. Biol. Chem. 272: 21201-21206). However, when the monovalent conjugate of peptide 3 was tested in the same assay, no activity could be detected when concentrations up to 100 nM were used in the assay.

What is to be claimed:

1. An immunoglobulin fusion protein useful for preparation of a pharmaceutical composition, said protein having the general formula:
B-(L)ₙ-(F) (I)
where B represents an at least one bioactive GLP-1 peptide, variant or derivative, F represents an antibody Fc comprising the structure (X)ₘ-(D)ₚ-CH2-CH3 where X represents any naturally occurring amino acid which may be incorporated and produced by standard molecular biological engineering techniques, where m is an integer from 0-20, D is a multimerizing or dimerizing domain, p is an integer from 0 to 1 and CH2 represents at least a portion of an immunoglobulin CH2 constant region which is joined to at least a portion of an immunoglobulin CH3 constant region; L represents a linker comprising a polymeric structure which is substantially nonimmunogenic and provides a flexible linkage between the bioactive moiety and F, where n can be the integers 0 or 1; and where n is 0, the linkage between B and F is a non-peptidyl covalent bond and when n is 1, the linkage between L and F is a non-peptidyl bond.

2. The protein of claim 1 of formula B—F and multimers thereof where the C-terminus of B is attached to the N-terminus of F or where the N-terminus of B is attached to the N-terminus of F and F lacks the dimerizing domain.

3. The protein of claim 1 of the formula B-L-F and multimers thereof wherein F is an Fc domain lacking the dimerizing domain and is attached by the N-terminus to L, and L is further attached at an alternated site to the C-terminus of B; or wherein F is a polypeptide as described capable of forming an Fc domain and is attached by the N-terminus to L, and L is further attached at an alternated site to the N-terminus of B.

4. The protein of claim 1 of the formula B¹—F —B² (IV) where B¹ and B² are the same or different GLP-1s or are conjugated to F via alternative sites on the same GLP-1 and where F has the dimerizing domain.

5. The protein of claim 1 of the formula B¹—L¹-F-L²-B² (V) where B¹ and B² are the same or different GLP-1s or are conjugated to L¹ and L², respectively, via alternative sites on B1 and B2 and where F has the dimerizing domain.

6. A protein according to claim 1, wherein the linkage between B and F or the linkage between L and F is selected from a hydrazine and a carbohydrazide group.

7. A method of preparing the protein of claim 6, wherein the hydrazine linkage is formed by reaction of a glyoxylyl-Fc (HCO—CO-Fc), a keto-Fc, or a simple aldehyde-Fc (HCO-Fc) which is reacted with an activated GLP-1 peptide having a hydrazine or hydrazide functionality to form a hydrazone at one or both of the N-termini of the Fc structure which may be further reduced to form the hydrazine linkage.

8. A method according to claim 7, wherein the glyoxylyl-Fc reacts with a linker which has been previously conjugated to the GLP-1 peptide, wherein the linker comprises a nucleophilic group selected from the group consisting of a hydrazine and a hydrazide moiety.

9. A method according to claim 8, wherein the glyoxylyl-Fc reacts with a hydrazine on linker (L) which further comprises a second reactive group which is not a hydrazine.

10. A protein according to any of claims 1 to 6, wherein the linker comprises at least one ethylene glycol unit.

11. A protein according to claim 1 wherein the polypeptide B has the sequence of SEQ ID NO: 2.

12. A protein according to claim 1 wherein B comprises a peptide selected from the group consisting of GLP-1 (7-36) of SEQ ID No. 1 or an analog thereof.

13. A protein according to claim 1 wherein D is at least a portion of an immunoglobulin hinge region.

14. A pharmaceutical composition comprising the protein of any of claims 1-6 in combination with a pharmaceutically acceptable carrier.

15. A method of treating a metabolic condition in a patient in need of such treatment comprising administering a therapeutically effective amount of a pharmaceutical composition comprising a protein of claim 1.

16. The method according to claim 14, wherein the condition is characterized by lack of glycemic control.

17. The method according to claim 14, wherein the condition is selected from the group consisting of Type 1 diabetes, a pre-diabetic condition, and Type 2 diabetes.


SEQUENCE LISTING (8 sequences)

SEQ ID NO: 1 - protein, 31 aa, Homo sapiens
  MOD_RES (31)..(31): AMIDATION
HAEGTFTSDVSSYLEGQAAKEFIAWLVKGRG

SEQ ID NO: 2 - protein, 31 aa, Artificial sequence
  Analogs of human GLP-1, chemically synthesized
HXXGXXXXXXXXXXXXXXXXXFXXXXXXXXX

SEQ ID NO: 3 - protein, 30 aa, Artificial sequence
  Artificial analogs of human GLP-1, chemically synthesized
HXXGTXXXXXSXYXEXXXXKXFXAWLXXGX

SEQ ID NO: 4 - protein, 38 aa, Heloderma suspectum
  VARIANT (2)..(2): Ser, Gly or Thr
HXXGTFTXDXSKQXEEEAVRLXXEXLKXGGPSSGAPPP

SEQ ID NO: 5 - protein, 8 aa, Artificial sequence
  Synthetic linking peptide
GGGKGGGG

SEQ ID NO: 6 - protein, 8 aa, Artificial sequence
  Synthetic linking peptide
GGGNGSGG

SEQ ID NO: 7 - protein, 9 aa, Artificial sequence
  Synthetic linking peptide
GGGCGGGGG

SEQ ID NO: 8 - protein, 5 aa, Artificial Sequence
  Synthetic linking peptide
GPNGG